(19) United States
(12) Patent Application Publication
(10) Pub. No.: US 2002/0077289 A1
(43) Pub. Date: Jun. 20, 2002

(54) Angiostatin and endostatin binding proteins and methods of use

(76) Inventors: Nicholas J. MacDonald, Chevy Chase, MD (US); Kim L. Sim, Gaithersburg, MD (US); John W. Holaday, Bethesda, MD (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 09/873,676

(22) Filed: Jun. 4, 2001

Related U.S. Application Data
(60) Provisional application No. 60/209,065, filed on Jun. 2, 2000.
(60) Provisional application No. 60/289,387, filed on May 8, 2001.

(51) Int. Cl. A61K 38/17; C07K 14/435

(52) U.S. Cl. 514/12; 530/350

(57) ABSTRACT

The present invention is related to compositions and methods for the modulation of angiogenesis. In particular, the present invention includes Angiostatin and Endostatin binding peptides and proteins and methods of using the same. The present invention identifies tropomyosin protein as an Endostatin binding protein and a laminin beta-1 chain as an Angiostatin binding protein. The present invention also provides methods of inhibiting angiogenesis in an individual comprising administering to the individual a tropomyosin binding compound and/or an actin cytoskeleton disrupting compound.

### CROSS REFERENCE TO RELATED APPLICATIONS

This application claims priority to U.S. Provisional Application Ser. No. 60/209,065 filed Jun. 2, 2000 and U.S. Provisional Application Ser. No. 60/289,387 filed May 8, 2001.

### FIELD OF THE INVENTION

The present invention is related to compositions and methods for the modulation of angiogenesis. In particular, the present invention includes Angiostatin and Endostatin binding peptides and proteins and methods of using the same.

### BACKGROUND OF THE INVENTION

As used herein, the term “angiogenesis” means the generation of new blood vessels into a tissue or organ. Under normal physiological conditions, humans or animals undergo angiogenesis only in very specific and restricted situations. For example, controlled angiogenesis is observed in wound healing, fetal and embryonal development and formation of the corpus luteum, endometrium and placenta. However, angiogenesis also occurs under abnormal or undesired conditions such as during tumor development, growth and metastasis. This type of angiogenesis may also be referred to as uncontrolled angiogenesis.

Both controlled and uncontrolled angiogenesis are thought to proceed in a similar manner. Endothelial cells and pericytes surrounded by a basement membrane form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

Persistent, unregulated angiogenesis occurs in a multiplicity of disease states, tumor metastasis and abnormal growth by endothelial cells. The diverse pathological disease states in which unregulated angiogenesis is present have been grouped together as angiogenesis-dependent or angiogenesis-related diseases. The hypothesis that tumor growth is angiogenesis-dependent was first proposed in 1971 by M. Judah Folkman. (Folkman J., N. Engl. Jour. Med. 285:1182-1186 (1971)). In its simplest terms the hypothesis states: “Once tumor ‘take’ has occurred, every increase in tumor cell population must be preceded by an increase in new capillaries converging on the tumor.” Tumor “take” is currently understood to indicate a pre-vascular phase of tumor growth in which a population of tumor cells occupying a few cubic millimeters volume, and not exceeding a few million cells, can survive on existing host microvessels. Expansion of tumor volume beyond this phase requires the induction of new capillary blood vessels.

Several molecules have been discovered that inhibit angiogenesis, inhibit tumor growth, cause regression of primary tumors, and/or inhibit metastasis of primary tumors. These molecules are called antiangiogenic agents. One of these antiangiogenic agents has been termed Endostatin. Endostatin is an antiangiogenic fragment of a C-terminal non-collagenous region of a collagen protein and is described in U.S. Pat. No. 5,854,205. Another antiangiogenic agent has been termed Angiostatin. Angiostatin is an antiangiogenic kringle region fragment of a plasminogen protein and is described in U.S. Pat. No. 5,639,725.

Although it has been shown that Endostatin and Angiostatin are potent inhibitors of angiogenesis, and can therefore be used for the treatment of angiogenesis-related diseases such as cancer, what is needed in the art is the identification of Endostatin and Angiostatin binding proteins and peptides. The natural substrate for plasminogen, the precursor of Angiostatin, is fibrin. Several studies have shown that fragments of plasminogen containing intact kringle domains bind to fibrin. For instance, Wu et al. showed that a plasminogen fragment containing kringles 1-3 binds to fibrin but with affinity less than a fragment containing kringles 1-5 (J. Biol. Chem., 265:19658-64 (1990)). Wu et al. also showed that kringle 4 does not bind to fibrin. The binding of plasminogen to fibrin involves interactions with lysines on fibrin and can be inhibited by lysine analogues. Only kringle 3 appears to be devoid of a lysine-binding motif (Cao et al., J. Biol. Chem., 271:29461-29467 (1996)), however, the plasminogen-binding sites on fibrin have not yet been identified (Weisel et al., J. Mol. Biol., 235:1117-1135(1994)). More importantly, it has not been determined whether Angiostatin inhibits angiogenesis via an interaction with fibrin or whether another Angiostatin binding protein is involved.

Identification of Endostatin and Angiostatin binding proteins would allow for the further elucidation of the mechanism of action of Endostatin and Angiostatin. Further elucidation of the mechanism of action of Endostatin and Angiostatin would allow for the creation of Endostatin and Angiostatin mimetics and for the creation of compositions and methods for the inhibition of angiogenesis that in essence “by-pass” the point of action of Endostatin and Angiostatin.

### SUMMARY OF THE INVENTION

The present invention includes Endostatin and Angiostatin binding proteins and peptides and nucleic acids that encode Endostatin protein and Angiostatin protein binding peptides and proteins. In particular, the present invention identifies tropomyosin protein as an Endostatin binding protein and a beta-1 chain of laminin protein as an Angiostatin binding protein. The present invention further includes tropomyosin binding compounds and actin disrupting compounds that inhibit angiogenesis. These compounds may be, but are not limited to, mimetics of Endostatin and Angiostatin. As used herein, the term “mimetic” refers to a proteinaceous or chemical compound that functions in a manner similar to a Endostatin protein or Angiostatin protein. A mimetic of Endostatin inhibits angiogenesis and also specifically binds to a tropomyosin protein. A mimetic of Angiostatin inhibits angiogenesis and specifically binds to a laminin beta-1 chain.

The methods of the present invention include methods of increasing angiogenesis in an individual comprising administering an angiogenesis increasing amount of an Endostatin binding protein or peptide or an Angiostatin binding protein or peptide. The present invention still further includes methods of inhibiting angiogenesis in an individual comprising administering to the individual an angiogenesis inhibiting amount of a tropomyosin binding compound. A tropomyosin binding compound includes, but is not limited to, an Endostatin mimetic. The present invention includes methods of inhibiting angiogenesis in an individual comprising administering to the individual an angiogenesis inhibiting amount of a laminin beta-1 chain binding compound. A laminin beta-1 chain binding compound includes, but is not limited to, an Angiostatin mimetic. The present invention also includes methods of inhibiting angiogenesis comprising administering to the individual a composition comprising an angiogenesis inhibiting amount of an actin cytoskeleton disrupting compound or a composition comprising antiangiogenic actin-binding molecules. In a preferred embodiment of the present invention, the actin disrupting compound is a tropomyosin binding compound. In an alternatively preferred embodiment, the present invention comprises Endostatin and a tropomyosin isoform as an antiangiogenic actin-binding composition.

The present invention also encompasses nucleotide sequences encoding peptides and proteins that bind angiogenesis-related peptides and proteins, as well as expression vectors containing nucleotide sequences encoding such binding peptides and proteins, and cells containing one or more expression vectors containing nucleotide sequences encoding such peptides and proteins. The present invention further encompasses gene therapy methods whereby nucleotide sequences encoding angiogenesis-related protein binding peptides and proteins are introduced into a patient to modify in vivo Angiostatin protein or Endostatin protein levels.

The present invention also includes diagnostic methods and kits for detection and measurement of peptides and proteins that bind angiogenesis-related proteins in biological fluids and tissues, and for localization of such peptides and proteins in tissues and cells. The diagnostic method and kit can be in any configuration well known to those of ordinary skill in the art.

The present invention includes peptides and proteins that bind Angiostatin protein or Endostatin protein and cause the transmission of an appropriate signal to a cell and act as agonists or antagonists of angiogenesis.

In addition, the present invention includes fragments of proteins that bind angiogenesis-related proteins, and analogs thereof, that can be labeled isotopically, or with other molecules or proteins, for use in the detection and visualization of angiogenesis-related protein binding sites with techniques, including, but not limited to, positron emission tomography, autoradiography, flow cytometry, radioreceptor binding assays, and immunohistochemistry.

The peptides and analogs of the present invention also act as agonists and antagonists for Angiostatin protein or Endostatin protein receptors, thereby enhancing or blocking the biological activity of Angiostatin protein or Endostatin protein. Such peptides and proteins are used in the isolation of Angiostatin protein or Endostatin protein receptors.

The present invention includes molecular probes for the ribonucleic acid and deoxyribonucleic acid involved in transcription and translation of angiogenesis-related protein binding peptides and proteins. These molecular probes provide means to detect and measure angiogenesis-related protein biosynthesis in tissues and cells.

Accordingly, it is an object of the present invention to provide compositions and methods comprising peptides and proteins that bind angiogenesis-related peptides and proteins.

It is another object of the present invention to provide compositions and methods for treating diseases and processes that are mediated by angiogenesis.

It is yet another object of the present invention to provide diagnostic or prognostic methods and kits for detecting the presence and amount of angiogenesis-related protein binding peptides in a body fluid or tissue.

It is yet another object of the present invention to provide compositions and methods for treating diseases and processes that are mediated by angiogenesis including, but not limited to, hemangioma, solid tumors, blood borne tumors, leukemia, metastasis, telangiectasia, psoriasis, scleroderma, pyogenic granuloma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, diabetic neovascularization, macular degeneration, wound healing, peptic ulcer, Helicobacter related diseases, fractures, keloids, vasculogenesis, hematopoiesis, ovulation, menstruation, placentation, and cat scratch fever.

It is another object of the present invention to provide compositions and methods for treating or repressing the growth of a cancer.

Another object of the present invention to provide compositions and methods for the detection or prognosis of cancer.

It is another object of the present invention to provide compositions and methods for use in visualizing and quantitating sites of Angiostatin protein or Endostatin protein binding in vivo and in vitro.

It is yet another object of the present invention to provide compositions and methods for use in detection and quantification of Angiostatin protein or Endostatin protein biosynthesis.

Another object of the present invention to provide receptors that bind angiogenesis-related proteins, such as Angiostatin protein or Endostatin protein.

Yet another object of the present invention is to identify proteins, and fragments thereof, that interact and regulate the activity of angiogenesis-related proteins such as Angiostatin protein or Endostatin protein.

Still another object of the present invention is to provide proteins, and fragments thereof, that are involved in the transport of angiogenesis-related proteins such as Angiostatin protein or Endostatin protein.

Another object of the present invention is to provide proteins, and fragments thereof that function as substrates through which angiogenesis-related proteins exert their activities.

It is yet another object of the present invention to provide a therapy for cancer that has minimal side effects.

Yet another object of the present invention is to provide methods and compositions comprising proteins and peptides, that bind Angiostatin protein comprising laminin protein, and/or proteins and peptides that bind Endostatin protein comprising tropomyosin.

Another object of the present invention is to provide methods and compositions comprising proteins and peptides, that bind Endostatin protein comprising human tropomyosin isoforms (hTMs) such as hTM1, hTM2, hTM3, hTM4, hTM5, hTM5a, hTM5b, hTMsma.

Still another object of the present invention is to provide methods and compositions comprising peptides, that bind Angiostatin protein and/or Endostatin protein, linked to a cytotoxic agent for treating or repressing the growth of a cancer.

Yet another object of the present invention is to provide methods and compositions comprising fusion proteins comprising peptides or proteins that bind Angiostatin protein and/or Endostatin protein.

It is another object of the present invention to provide methods and compositions comprising fusion proteins comprising laminin and tropomyosin for binding and regulating Angiostatin protein and/or Endostatin protein.

Another object of the present invention is to provide methods and compositions for targeted delivery of angiogenesis-related protein compositions to specific locations.

Yet another object of the invention is to provide compositions and methods useful for gene therapy for the modulation of angiogenic processes.

These and other objects, features and advantages of the present invention will become apparent after a review of the following detailed description of the disclosed embodiments and the appended claims.

### BRIEF DESCRIPTION OF THE FIGURES

FIG. 1 shows a schematic representation of the structure of human plasminogen and its kringle fragments. Human plasminogen is a single chain protein containing 791 amino acids with one site of N-linked glycosylation at Asn²⁸⁹. The non-protease region of human plasminogen consists of the N-terminal 561 amino acids existing in five separate domains, termed kringles as shown in circles (K1, K2, K3, K4 and K5), along with the protein sequences (or with the amino acids) that separate these structures. Each triple disulfide bonded kringle contains approximately 80 amino acids. Angiostatin covers the first 4 of these kringle domains (K1-4), kringle 3 (K1-3) and kringle 4 (K4) are obtained by digestion of human plasminogen with elastase. The rest of the kringle fragments are recombinant proteins expressed in E. coli. ‘SS’ signifies signal sequence. ‘PA’ signifies pre-activation protein.

FIG. 2 shows the preferred amino acid sequence of human Angiostatin protein (SEQ ID NO: 1).

FIG. 3 shows the preferred amino acid sequence of human Endostatin protein (SEQ ID NO: 2).

FIG. 4 provides graphs demonstrating binding preference of linear peptides (selected by display technology) for Angiostatin protein over plasminogen.

FIG. 5 provides graphs demonstrating binding preference of cyclized peptides (selected by display technology) for Angiostatin protein over plasminogen.

FIG. 6A provides the complete amino acid sequence of laminin protein (SEQ ID NO:113), and

FIG. 6B provides the complete nucleotide sequence of laminin protein (SEQ ID NO:114).

FIG. 7 shows the three motifs of laminin protein corresponding to Angiostatin protein selected peptides in a single 60 amino acid long region.

FIG. 8 is a graph showing the results of an ELISA assay demonstrating the interaction of tropomyosin and Endostatin protein.

FIG. 9 provides the results of an in vivo experiment for assessing the interaction of peptide E37 and Endostatin protein.

FIG. 10 provides a graph showing the results of an in vivo experiment for assessing the interaction of peptide E37 and Endostatin protein as the mean, standard deviation, T/C and the results of a 2-tailed T-test.

FIG. 11 provides a graph showing the results of an in vivo experiment regarding the inhibition of the anti-metastatic activity of Endostatin protein by an E37 peptide.

FIG. 12 provides a schematic demonstrating that the putative Endostatin-binding site of hTM3 is not present in all isoforms of tropomyosin. 50 micrograms (μg) each of hTM3 expressing λ21-1 and control E. coli lysate; and 250 ng each of purified cardiac (human) and muscle (rabbit and chicken) tropomyosin proteins were resolved using SDS-PAGE under non-reducing conditions and immunoblotted. Blots were reacted with TM311 anti-tropomyosin ascites fluid (A), and anti-E37 sera (B), respectively. Although the TM311 anti-tropomyosin antibody recognized all isoforms of tropomyosin tested, the anti-E37 sera recognized only the bacterially expressed hTM3 of the λ21-1 lysate indicating that not all isoforms of tropomyosin contain the putative Endostatin-binding site. The stars (*) mark the position of the ˜49 and 43 kDa proteins detected in the λ221-1 lysate by both TM311 and anti-E37 sera.

FIG. 13 provides a graph showing that rhTM3 binds rhEndostatin in vitro. The wells of a 96-well plate were coated with either 5 micrograms/milliliter (μg/ml) of rhEndostatin (closed symbols) or BSA (open symbols) and incubated with increasing amounts of control E. coli lysate (circles) or tropomyosin expressing λ21-1 bacterial lysate (squares). The amount of tropomyosin binding to each well was determined spectrophotometrically following the addition of the TM311 anti-tropomyosin ascites fluid, alkaline phosphatase conjugated anti-mouse IgG and substrate. Error bars indicate standard deviation of the mean.

FIGS. 14(A-C) provide graphs demonstrating the kinetics of the interaction of soluble rhEndostatin with immobilized rhTM3. (A), 0, 10, 20, 40, 80 and 160 micromolar (μM) of rhEndostatin were injected at 5 microliters per minute (μl/minute) for 960 seconds through a biosensor flow cell that had been activated and blocked as a control for bulk refractive index and subsequently through a flow cell to which 3200 response units (RU) of rhTM3 had been immobilized. The binding kinetics were recorded and the differences between the two curves plotted for each rhEndostatin concentration. (B), 0, 11.5, 23 and 46 micromolar (μM) of rhAngiostatin were injected at 5 microliters per minute (μl/minute) for 900 seconds over a flow cell containing 3200 RU of rhTM3 as above. Samples were run in duplicate, the data corrected for bulk shift and the average of the 2 curves plotted. (C), Injection of 5 micromolar (μM) of rhEndostatin at 5 microliters per minute (μl/minute) for 240 seconds in the presence of 0, 6, 15, 30 and 45 micromolar (μM) of soluble rhTM3 over a flow cell containing 2000 RU of rhTM3. Samples were run in duplicate, the reference cell values subtracted and the average of each of the 2 curves plotted. The spikes in RU seen at the beginning and end of each injection are due to lag in flow caused by the rhTM3 and reference cells being non-adjacent.

FIGS. 15(A-E) provide schematics demonstrating that rhEndostatin-binding and tropomyosin co-localize to the microfilaments of human endothelial cells. (A), HUVECs, (B), HAECs and (C), HMVECs were incubated in the presence of 40 micrograms per milliliter (μg/ml) Alexa 488-labeled rhEndostatin and photographed at 100× magnification under Alexa 488 (green) excitation wavelength. (D) and (E), HUVECs were incubated simultaneously in anti-tropomyosin TM311 ascites fluid and 40 micrograms per milliliter (μg/ml) Alexa 488-labeled rhEndostatin followed by incubation in Alexa 594 conjugated goat anti-mouse IgG (H+L). The same field was photographed under Alexa 488 (green) and Alexa 594 (red) excitation wavelengths at 100× magnification.

FIGS. 16(A-C) provide schematics demonstrating that the inhibition of rhEndostatin's binding to HUVEC mircofilaments by E37 peptide implicates tropomyosin's role. HUVECs were incubated in the presence of 40 micrograms per milliliter (μg/ml) Alexa 488-labeled rhEndostatin alone (A), or in the presence of a 50-fold molar excess of control peptide (B), or E37 peptide (C). Cells were photographed using the same exposure times at 100x magnification under Alexa 488 (green) excitation wavelength.

FIG. 17 provides a graph demonstrating that the E37 peptide inhibits the anti-metastatic activity of rhEndostatin. C57BL/6J mice were injected via the tail vein with 5×10⁴ B16-BL6 melanoma cells on day 0. On day 3, groups of five mice received daily subcutaneous doses of either buffer control, 1.5 nmol (1.5 mg/kg/day) of rhEndostatin in combination with 0, 15, 75 or 375 nmol of control or E37 peptide, or control or E37 peptide alone for eleven days. All mice were sacrificed on day 14, and the number of pulmonary surface metastases in each animal counted. Error bars indicate standard deviation of the mean.

FIG. 18 provides sequences for hTM3 (SEQ ID NO: 119), and fragments of interest exon 6 (SEQ ID NO: 116) and exon 9 (SEQ ID NO: 117).

### DETAILED DESCRIPTION

The following description includes the best presently contemplated mode of carrying out the invention. This description is made for the purpose of illustrating the general principles of the inventions and should not be taken in a limiting sense. All publications, references, applications and patents listed or cited herein are incorporated by reference in their entirety.

The present invention includes Endostatin and Angiostatin binding proteins and peptides and nucleic acids that encode Endostatin protein and Angiostatin protein binding peptides and proteins. In particular, the present invention identifies tropomyosin protein as an Endostatin binding protein and laminin protein as an Angiostatin binding protein. In particular, the present invention includes methods and compositions comprising tropomyosin isoforms that contain the E37 mimotope (that include but are not limited to human tropomyosin 3 (hTM3))for use in the regulation of angiogenesis. The present invention also includes methods of regulating angiogenesis in an individual comprising administering an angiogenesis modifying amount of an Endostatin binding protein or peptide or an Angiostatin binding protein or peptide.

The present invention further includes tropomyosin binding compounds and actin cytoskeleton disrupting compounds that inhibit angiogenesis. The tropomyosin binding compound includes, but is not limited to, an Endostatin mimetic. The actin cytoskeleton disrupting compound includes, but is not limited to, a tropomyosin binding compound and an Endostatin mimetic. The present invention still further includes methods of inhibiting angiogenesis in an individual, comprising of administering to the individual an angiogenesis inhibiting amount of a tropomyosin binding compound. In a specific embodiment, the present invention includes methods and compositions for regulating angiogenesis comprising Endostatin and/or Endostatin mimetics that bind hTM3. The present invention also includes methods of inhibiting angiogenesis comprising, administering to the individual an angiogenesis inhibiting amount of an actin cytoskeleton disrupting compound. As used herein, the term “inhibiting angiogenesis” refers to a reduction or inhibition of the growth of blood vessels into tissues, organs, or unvascularized or vascularized tumors. The ability of a compound to inhibit angiogenesis may be demonstrated in vitro using methods well known to those of skill in the art such as the BCE assay and the CAM assay as described in U.S. Pat. No. 5,854,205.

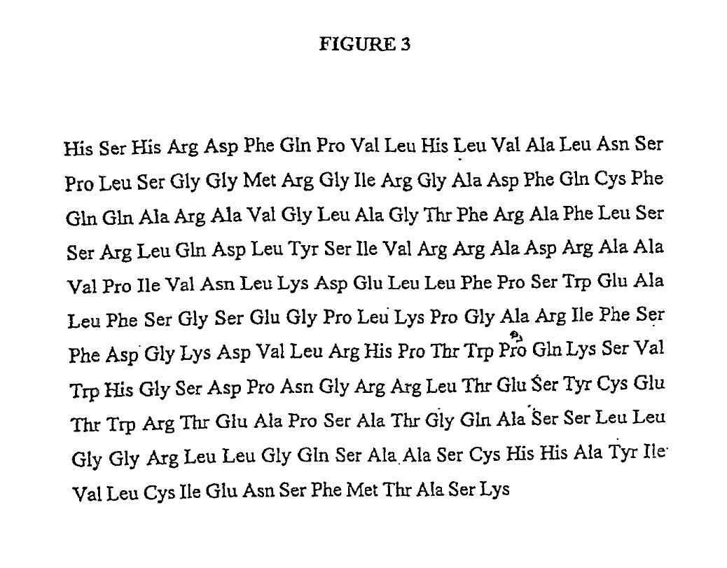

As used herein, the term “Endostatin” refers to an antiangiogenic fragment of a most C-terminal non-collagenous region of a collagen protein. It is to be understood that the terms “Endostatin” and “Endostatin protein” are equivalent and interchangeable. In a preferred embodiment, the C-terminal non-collagenous region is an NC1 region. The collagen protein may be any collagen protein (any member of the collagen family of proteins), but is preferably a non-fibrillar collagen protein, and more preferably a collagen XVIII, a collagen XV or a collagen IV. In one embodiment, an Endostatin is a fragment of an approximately 35 kDa C-terminal non-collagenous region of collagen XVIII. In a further embodiment, an Endostatin is a fragment of an approximately 35 kDa C-terminal non-collagenous region of collagen XVIII having a molecular weight of between approximately 18 kDa and 20 kDa. In another embodiment, an Endostatin is a fragment of an approximately 35 kDa C-terminal non-collagenous region of collagen XVIII and comprises an amino acid sequence identical or substantially homologous to amino acids 1105 to 1124 of mouse collagen alpha 1 type XVIII. In another embodiment, an Endostatin is a fragment of an approximately 35 kDa C-terminal non-collagenous region of collagen XVIII and comprises an amino acid sequence identical or substantially homologous to amino acids 1132 to 1151 of human collagen alpha 1 type XVIII. In yet another embodiment, an Endostatin has an N-terminal amino acid sequence of HTHQDFQPVLHLVALNTPLS (SEQ ID NO:115). One preferred amino acid sequence for human Endostatin protein is shown in FIG. 3 and SEQ ID NO:2. The corresponding cDNA sequence of this preferred Endostatin is shown in SEQ ID NO 30.

As used herein, the term “Angiostatin” refers to an antiangiogenic kringle region fragment of a plasminogen protein. It is to be understood that the terms “Angiostatin” and “Angiostatin protein” are equivalent and interchangeable. It is also to be understood that the term “Angiostatin” encompasses kringle region fragments of plasminogen consisting of the N-terminal sequence proceeding a kringle 1 region, a kringle 2 region, a kringle 3 region, a kringle 4 region, a kringle 5 region, inter-kringle regions, and antiangiogenic fragments and combinations thereof In one embodiment, Angiostatin comprises approximately kringle regions 1 through 4 of a plasminogen molecule and has a molecular weight of between approximately 38 kDa and 45 kDa as determined by reducing polyacrylamide gel electrophoresis. In an alternative embodiment, Angiostatin comprises approximately kringle regions 1 through 5. A preferred amino acid sequence for human Angiostatin is shown in FIG. 3 (or SEQ ID NO:1).

The amino acid sequence of the complete murine plasminogen molecule is shown in SEQ ID NO:81.

The cDNA sequence for human angiostatin protein is provided as SEQ ID NO: 29.

As used herein, the term “angiogenesis-related protein” refers to Angiostatin protein and Endostatin protein, and antiangiogenic fragments and homologs thereof. The term “angiogenesis-related protein” includes proteins that are animal or human in origin and also includes proteins that are made synthetically by chemical reaction, or by recombinant technology in conjunction with expression systems. Angiogenesis-related proteins can be isolated from body fluids including, but not limited to, serum, urine and ascites, or synthesized by chemical or biological methods (including cell culture, recombinant gene expression, peptide synthesis). The proteins may also be obtained by in vitro enzymatic catalysis of plasminogen or plasmin to yield active Angiostatin protein, or of collagen to yield active Endostatin protein. Recombinant techniques include gene amplification from DNA sources using the polymerase chain reaction (PCR), and gene amplification from RNA sources using reverse transcriptase/PCR. General references for methods that can be used to perform the various PCR and cloning procedures described herein can be found in Molecular Cloning: A Laboratory Manual (Sambrook et al., eds. Cold Spring Harbor Lab Publ. 1989, latest edition).

As used herein, the term “binding peptide” refers to Endostatin binding peptides and Angiostatin binding peptides. The term “binding peptide” includes active fragments, homologs and derivatives Endostatin binding peptides and Angiostatin binding peptides that bind Endostatin protein and Angiostatin protein, respectively. Exemplary binding peptides, their amino acid sequences (SEQ ID NOS:3-28, SEQ ID NOS:31-42, SEQ ID NOS:82-113 and SEQ ID NO:119.), and their experimental abbreviations are set forth in the description below. Using the Endostatin and Angiostatin binding peptides described herein, Endostatin and Angiostatin binding proteins can be identified. The term “binding protein” is used herein to refer to Endostatin binding proteins and Angiostatin binding proteins that either comprise the binding peptides described herein or have a three-dimensional conformational epitope that corresponds to the binding peptides described herein. For example, the present invention describes Endostatin binding peptide E37 shown in Table 3 and also describes that Endostatin binding peptide E37 represents a three-dimensional conformational epitope of tropomyosin protein which is an Endostatin binding protein.

It is to be understood that the binding peptides and proteins herein are other than naturally occurring immunoglobulin antibody molecules. It is also to be understood that the binding peptides and binding proteins can be animal or human in origin. Although the Endostatin and Angiostatin binding peptides described in more detail below were identified using phage display technology, the binding peptides and binding proteins can also be produced synthetically by chemical reaction or by recombinant techniques in conjunction with expression systems.

The terms “a”, “an” and “the” as used herein are defined to mean “one or more” and include the plural unless the context is inappropriate. The term “bodily fluid,” as used herein, includes, but is not limited to, saliva, gingival secretions, cerebrospinal fluid, gastrointestinal fluid, mucous, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, vitreal fluid, and nasal secretions. The phrases “isolated” or “biologically pure” refer to material which is substantially or essentially free from components which normally accompany it as found in its native state.

It is to be understood that the present invention is contemplated to include any binding peptide, binding protein, or Angiostatin or Endostatin protein derivative. A binding peptide, binding protein, or Angiostatin or Endostatin protein derivative includes a protein or peptide having a modified sequence in which one or more amino acids in the original sequence or subsequence have been substituted with a naturally occurring amino acid residue or amino acid residue analog (also referred to as modified amino acid). Suitable binding peptide, binding protein, and Angiostatin or Endostatin protein derivatives have modified sequences which are substantially homologous to the amino acid sequence of a binding peptide, a binding protein, and an Endostatin protein, respectively.

An “amino acid residue” is a moiety found within a protein or peptide and is represented by —NH—CHR—CO—, wherein R is the side chain of a naturally occurring amino acid. When referring to a moiety found within a peptide, the terms “amino acid residue” and “amino acid” are used interchangeably. An “amino acid residue analog” includes D or L configurations having the following formula: —NH—CHR—CO—, wherein R is an aliphatic group, a substituted aliphatic aromatic group, a benzyl group, a substituted benzyl group, an aromatic group or a substituted aromatic group and wherein R does not correspond to the side chain of a naturally occurring amino acid.

Suitable substitutions for amino acid residues in the sequence of the binding peptides and binding proteins described herein include conservative substitutions that result in peptide and protein derivatives that bind angiogenesis-related proteins. Suitable substitutions for amino acid residues in the sequence of the Endostatin proteins described herein include conservative substitutions that result in antiangiogenic Endostatin protein derivatives. A conservative substitution is a substitution in which the substituting amino acid (naturally occurring or modified) is structurally related to the amino acid being substituted. “Structurally related” amino acids are approximately the same size and have the same or similar functional groups in the side chains.

Provided below are groups of naturally occurring and modified amino acids in which each amino acid in a group has similar electronic and steric properties. Thus, a conservative substitution can be made by substituting an amino acid with another amino acid from the same group. It is to be understood that these groups are non-limiting and that additional modified amino acids could be included in each group.

Group I includes leucine, isoleucine, valine, methionine and modified amino acids having the following side chains: ethyl, n-propyl n-butyl. Preferably, Group I includes leucine, isoleucine, valine and methionine.

Group II includes glycine, alanine, valine and a modified amino acid having an ethyl side chain. Preferably, Group II includes glycine and alanine.

Group III includes phenylalanine, phenylglycine, tyrosine, tryptophan, cyclohexylmethyl, and modified amino residues having substituted benzyl or phenyl side chains. Preferred substituents include one or more of the following: halogen, methyl, ethyl, nitro, —NH₂, methoxy, ethoxy and —CN. Preferably, Group III includes phenylalanine, tyrosine and tryptophan.

Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, —CO—NH-alkylated glutamine or asparagine (e.g., methyl, ethyl, n-propyl and iso-propyl) and modified amino acids having the side chain —(CH₂)₃—COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, methyl aspartate, ethyl aspartate, benzyl aspartate and methyl glutamate, ethyl glutamate and benzyl glutamate, glutamine and asparagine.

Group V includes histidine, lysine, ornithine, arginine, N-nitroarginine, β-cycloarginine, γ-hydroxyarginine, N-amidinocitruline and 2-amino-4-guanidinobutanoic acid, homologs of lysine, homologs of arginine and homologs of ornithine. Preferably, Group V includes histidine, lysine, arginine and ornithine. A homolog of an amino acid includes from 1 to about 3 additional or subtracted methylene units in the side chain.

Group VI includes serine, threonine, cysteine and modified amino acids having C₁-C₅ straight or branched alkyl side chains substituted with —OH or —SH, for example, —CH₂CH₂OH, —CH₂CH₂CH₂OH or —CH₂CH₂OHCH₃. Preferably, Group VI includes serine, cysteine or threonine.

In another aspect of the present invention, suitable substitutions for amino acid residues in the amino acid sequences described herein include “severe substitutions” that result in binding peptides and binding proteins that bind to angiogenesis-related proteins. Suitable substitutions for amino acid residues in the amino acid sequences described herein also include “severe substitutions” that result in Angiostatin or Endostatin protein derivatives that are antiangiogenic. Severe substitutions that result in binding peptide and binding protein derivatives and antiangiogenic Angiostatin or Endostatin protein derivatives are much more likely to be possible in positions that are not highly conserved than at positions that are highly conserved. A “severe substitution” is a substitution in which the substituting amino acid (naturally occurring or modified) has significantly different size and/or electronic properties compared with the amino acid being substituted. For example, the side chain of the substituting amino acid can be significantly larger (or smaller) than the side chain of the amino acid being substituted and/or can have functional groups with significantly different electronic properties than the amino acid being substituted.

Examples of severe substitutions of this type include the substitution of phenylalanine or cyclohexylmethyl glycine for alanine, isoleucine for glycine, a D amino acid for the corresponding L amino acid or NH—CH[(—CH₂)₅—COOH]—CO— for aspartic acid. Alternatively, a functional group may be added to the side chain, deleted from the side chain or exchanged with another functional group. Examples of severe substitutions of this type include adding an amine or hydroxyl, carboxylic acid to the aliphatic side chain of valine, leucine or isoleucine, exchanging the carboxylic acid in the side chain of aspartic acid or glutamic acid with an amine or deleting the amine group in the side chain of lysine or ornithine. In yet another alternative, the side chain of the substituting amino acid can have significantly different steric and electronic properties that the functional group of the amino acid being substituted. Examples of such modifications include tryptophan for glycine, lysine for aspartic acid and —(CH₂)₄COOH for the side chain of serine. These examples are not meant to be limiting.

The present invention encompasses homologs, orthologs and paralogs of Endostatin and Angiostatin binding peptides and proteins. Homologs are defined as proteins with substantial homology. “Substantial homology” exists between two amino acid sequences when a sufficient number of amino acid residues at corresponding positions of each amino acid sequence are either identical or structurally related such that a protein or peptide having the first amino acid sequence and a protein or peptide having the second amino acid sequence exhibit similar biological activities. Generally, there is substantial sequence homology among the amino acid sequences when at least 70%, more preferably at least 80%, and most preferably at least 90%, of the amino acids in the first amino acid sequence are identical to or structurally related to the second amino acid sequence. Homology is often measured using sequence analysis software, e.g., BLASTIN or BLASTP. The default parameters for comparing the two sequences (e.g., “Blast”-ing two sequences against each other) by BLASTIN (for nucleotide sequences) are reward for match=1, penalty for mismatch=−2, open gap=5, and extension gap=2. When using BLASTP for protein sequences, the default parameters are reward for match=0, penalty for mismatch=0, open gap=11, and extension gap=1. Additionally, paralogs are defined as proteins having non-identical amino acid sequences and similar functional characteristics, wherein the proteins are from the same species. Orthologs are defined as proteins having non-identical amino acid sequences and similar functional characteristics, wherein the proteins are from different species, but wherein the species have a common ancestral origin. Orthologs have at least 30% homology, more preferably at least 40% homology, and most preferably at least 50% homology among the amino acid sequences.

Unless defined otherwise, all technical and scientific terms used herein have the same meaning as commonly understood by one having ordinary skill in the art to which this invention belongs. Although other materials and methods similar or equivalent to those described herein can be used in the practice or testing of the present invention, the preferred compositions and methods are now described

The present invention includes Endostatin and Angiostatin binding peptides. The amino acid sequences of exemplary binding peptides are shown in SEQ ID NOS:3-28, SEQ ID NOS:31-42 SEQ ID NOS:82-113, and SEQ ID NO:119. The present invention also relates to proteins which share homologous sequences with the Endostatin and Angiostatin binding peptides. Such proteins include human possible global transcription activator, phosphopentomutase, ribonuclease RH precursor, soybean early nodulin, JNK activating kinase 1, IL-12 beta chain, glutathione reductase, soy bean trypsin inhibitor (kunitz), fibroblast growth factor-6, chemotaxis protein, annexin XI, WEE 1, RAS suppressor protein 1, ATP synthase gamma chain, thioredoxin, collagenese, glycoprotein B-1 precursor, dehydroquinate dehydratase, complement component C8 beta chain, omithine decarboxylase antizyme, adenylate cyclase, and ATP synthase, alpha chain.

The homology searches for the present invention were conducted using the FASTA sequence similarity search. The peptide sequences were queried against the SwissProt database using the default setting (ktup 2, and BLOSUM50 for the scoring matrix). The searches were conduced using the world wide web at site http://www.fasta.genome.adjp/. as described by W. R. Pearson & D. J. Lipman, PNAS 85:2444-2448 (1988).

As described in further detail below, the binding peptides described herein have many uses. In particular, the binding peptides are used for the identification and isolation of the binding proteins described herein. One approach used to identify Endostatin and Angiostatin binding proteins is to use the binding peptide to generate antibodies. These anti-peptide antibodies recognize proteins with which Angiostatin protein or Endostatin protein interact. Example 6 describes the identification of tropomyosin protein as an Endostatin binding protein through the use of an antibody to the E37 binding peptide. The anti-peptide and anti-protein antibodies are also useful since these antibodies would affect the anti-angiogenic activity of the Angiostatin protein or Endostatin protein. Thus, these anti-peptide and anti-protein antibodies can be assayed for their ability to affect the activity of Angiostatin protein or Endostatin protein.

The anti-peptide and anti-protein antibodies may also be used to screen phage expression libraries such as a λgt11 expression library. Such an approach would enable the cloning of the cDNA corresponding to proteins that interact with Angiostatin protein or Endostatin protein. Once the cDNAs have been identified they may be produced using recombinant technology and tested alone and in combination with their target angiogenesis-related protein for their angiogenic or anti-angiogenic activity.

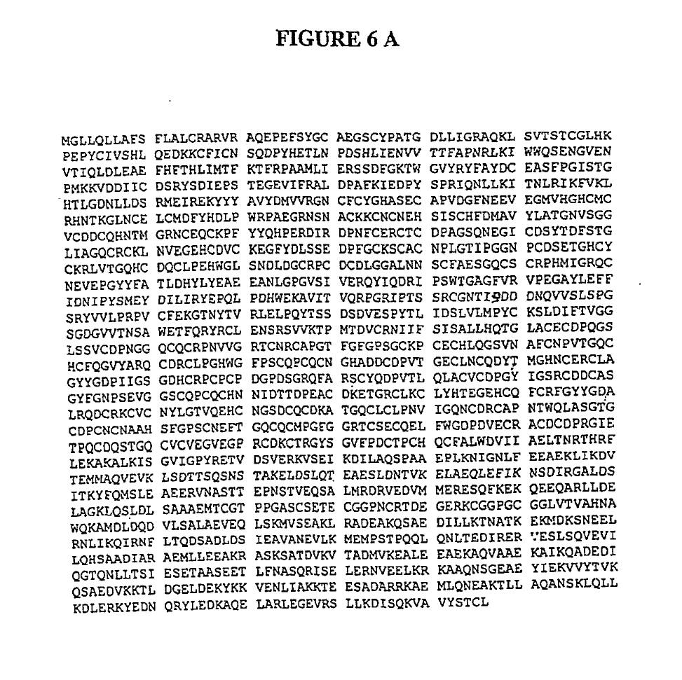

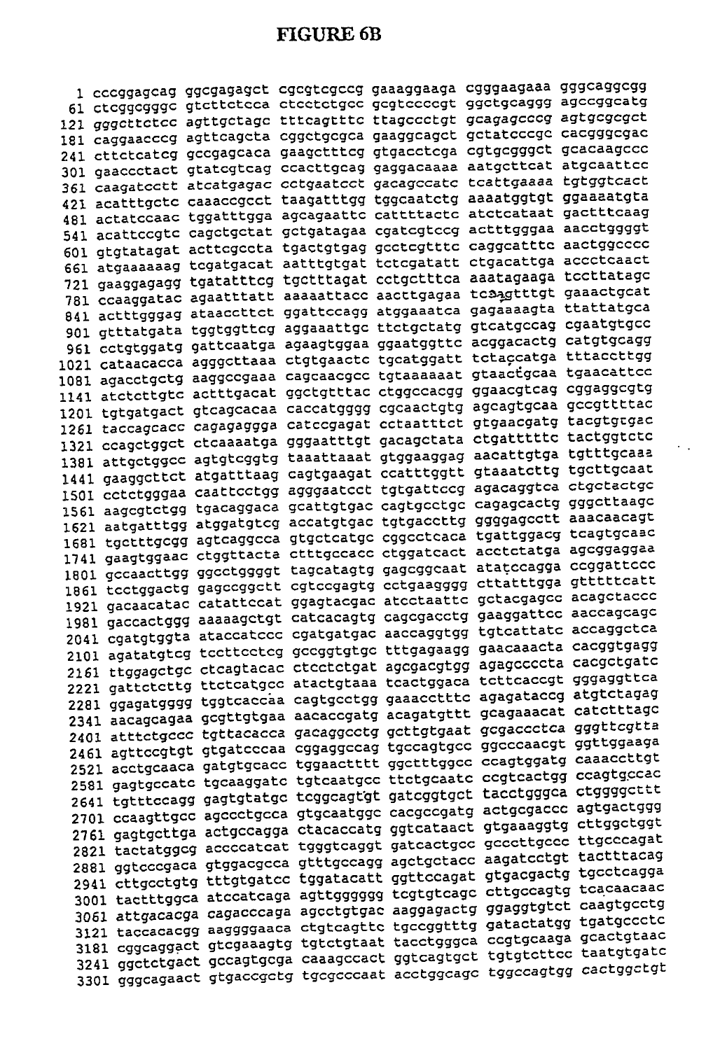

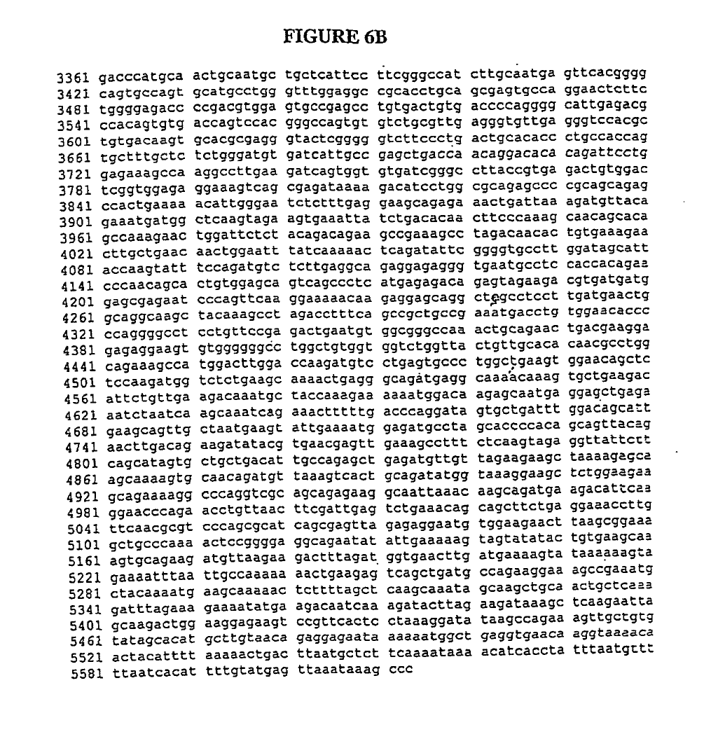

With regard to Angiostatin binding proteins in particular, the present invention is particularly related to basement membrane proteins including, but not limited to, laminin protein. As demonstrated in Example 11 below, as a result of sequence similarities between peptides that bind Angiostatin protein and laminin protein, laminin protein is considered to comprise a receptor, or a molecule closely related to a receptor, for Angiostatin protein. In addition, laminin protein may also be considered a protein that is involved in mediating the activity of Angiostatin protein. Based upon the findings of the present invention, it is believed that Angiostatin protein interacts with the beta-1 chain of laminin protein. Laminin is a basement membrane derived noncollagenous glycoprotein found in the extracellular matrix which binds type IV collagen, glycosaminoglycan and heparin, and is involved in the promotion of cellular adhesion. Laminin protein is a heterotrimeric molecule with an alpha, beta and gamma chain. The amino acid sequence of laminin protein is set forth in FIG. 6A (SEQ ID NO: 113) and the nucleotide sequence is set forth in FIG. 6B (SEQ ID NO: 114).

The present invention also includes methods of modulating and/or inhibiting angiogenesis, especially angiogenesis that is related to tumor growth, comprising administering laminin protein to an individual. Methods of inhibiting angiogenesis include methods comprising administering a laminin protein via gene therapy whereby the transfected cell has increased laminin protein cell surface expression. The present invention also includes methods of inhibiting angiogenesis comprising administering one or more alpha, beta or gamma chains of a laminin protein, or a combination thereof, via gene therapy whereby the transfected cell has increased cell surface expression of alpha, beta and/or gamma chains of a laminin protein, or a combination thereof. Additionally included in the present invention are methods of increasing angiogenesis comprising administering soluble laminin protein to an individual. The soluble laminin protein binds to the circulating Angiostatin protein, and therefore, increases angiogenesis in the individual.

With regard to Endostatin binding proteins, the present invention is related to a tropomyosin protein. Tropomyosins are a large family of proteins: at least 20 different isoforms exist that are generated by alternative splicing of a multigene family (Pittenger et al. Curr Opin Cell Biol 6(1)96-104 (1994); Lin et al. Int Rev Cytol 170:1-38 (1997) and references therein). They bind the alpha-helical groove of actin filaments to stabilize actin in the polymerized state directly influencing the integrity of microfilaments and thus play a role regulating reorganization of the actin cytoskeleton. Tropomyosins have been identified in organisms as diverse as yeast and man, and are core components of the cell cytoskeleton. Many vertebrate non-muscle cells express between five and eight isoforms of tropomyosin in a tissue-specific_manner, leading to speculation that tropomyosin isoforms may have evolved to perform specific functions in the microfilaments of non-muscle cells. Indeed, mutational analysis indicates that tropomyosin isoforms have distinct functions and that they play important roles in a variety of cellular functions, including contraction, cytokinesis, intracellular transport, secretion, motility, morphogenesis and cell transformation (Lin et al. and references therein). The interation of tropomyosin with Endostatin and subsequent consequences was unknown prior to the discoveries by the present inventors as detailed herein.

As used herein the term ‘tropomyosin’ refers to all isoforms of tropomyosin.

The molecular mechanism of Endostatin's antiangiogenic activity is unknown. To elucidate Endostatin's mode of action a phage-display library was screened to identify the binding domains of proteins with which Endostatin potentially interacts. Evidence is presented herein showing that human tropomyosin isoform 3 (hTM3) shares an epitope with an Endostatin-binding peptide, that rhEndostatin and hTM3 interact, in vitro and that rhEndostatin protein binds tropomyosin-containing mircofilaments of endothelial cells. Also presented is a demonstration that the peptide mimotope of hTM3's Endostatin-binding site blocks the anti-tumor activity of rhEndostatin in vivo and suggest that Endostatin's anti-tumor activity results from an interaction with tropomyosin-containing microfilaments that leads to inhibition of microfilament function and an induction of apoptosis.

As demonstrated in the Examples below, tropomyosin is considered to comprise a receptor, or a molecule closely related to a receptor, for Endostatin protein. In addition, tropomyosin may also be considered a protein that is involved in mediating activity of Endostatin protein. Specifically, the 3 dimensional shape of the E37 peptide (SEQ ID NO:25) is similar to a (3 dimensional) region of the tropomyosin protein. Mimotopic homology may be characterized as ‘shape homology’. As is known by those skilled in the art, because of 3 dimensional folding of proteins, epitopes are not always comprised of linear stretches of protein sequence. Such epitopes can be mimicked by linear sequences whose shape corresponds to the shape of a non-linear epitope, such peptides are said to be mimotopes. An important finding of the present invention is that the E37 peptide comprises a mimotope of the actual ligand binding site of tropomyosin. Accordingly, the findings of the present invention surprisingly defines not only an Endostatin protein receptor, but also defines an actual ligand binding domain as represented by the E37 peptide.

As further detailed in the Examples, both in vitro and in vivo experiments demonstrate that tropomyosin comprises a receptor, or a molecule closely related to a receptor for Endostatin protein. Antibodies against the E37 peptide have shown that the E37 peptide and tropomyosin share a common epitope, and it is believed that by virtue of this epitope, Endostatin protein binds specifically to tropomyosin and that the anti-angiogenic activity of Endostatin protein is mediated via this interaction. As used herein, the term “binds specifically to” refers to a binding reaction wherein Endostatin protein binds preferentially to tropomyosin protein and does not bind in a significant amount to other proteins present in a random sample.

Notably, it is a surprising finding of the present invention that Endostatin protein binds hTM3, but does not bind human cardiac tropomyosin. There are over 20 different isoforms (types) of tropomyosin expressed by mammalian cells. Tropomyosins are components of the cell cytoskeleton, which bind the alpha-helical groove of actin filaments to stabilize actin in the polymerized state. Consequently, tropomyosins directly influence the integrity of microfilaments. In many vertebrate non-muscle cells, between five and eight isoforms of tropomyosin exist and are expressed in a tissue specific manner (Lin, J. J. et al., Int. Rev. Cytol., 170:1-38 (1997)). In particular, human fibroblasts express the following forms of tropomyosin proteins hTM1, hTM2, hTM3, hTM4, hTM5, hTM5a, hTM5b, hTMsma. The hTM2, hTM3, hTM5a, hTM5b, hTMsma tropomyosin proteins are encoded by the hTMa gene. The hTM1 tropomyosin protein is encoded by the hTMb gene. The hTM5 tropomyosin protein is encoded by the hTMnm gene. The hTM4 tropomyosin protein is encoded by the hTMpl gene. The hTM3 tropomyosin protein is commonly referred to as fibroblast tropomyosin, while the hTM1 tropomyosin protein is sometimes referred to as epithelial tropomyosin. It is believed that tropomyosin isoforms perform specific functions in the actin-based microfilaments of non-muscle cells.

Though not wishing to be bound by the following theories, it is believed that the interaction between Endostatin protein and hTM3 (and/or another non-muscle cell tropomyosin) targets the Endostatin protein to newly formed blood vessels instead of established blood vessels throughout the rest of the body. During the formation of new blood vessels (i.e., angiogenesis), the newly formed (or immature) blood vessels are relatively unstable (they can regress and be remodeled readily). As the blood vessels become more mature and stable, they associate with pericytes, which form a barrier around the endothelial cells. At some point during this process, the pericytes differentiate into fibroblasts. Endostatin protein may bind to pericytes expressing fibroblast tropomyosin, inhibit pericyte differentiation and channel these cells into the apoptotic pathway. There is some evidence that vessels associated with pericytes cannot regress and that this may explain why antiangiogenic compounds like Angiostatin protein and Endostatin protein target the newly formed vasculature of tumors and not the established blood vessels necessary for maintenance of vital organs. Additionally, there is also evidence that Endostatin protein binds to the surface of fibroblasts, and therefore, Endostatin protein may be acting via its interaction with tropomyosin protein expressed by fibroblasts.

The Examples that follow support the above theories. Specifically the examples demonstrate that Endostatin protein specifically binds to tropomyosin protein in vitro and these proteins co-localize in vivo. Additionally, Example 8 demonstrates that the E37 peptide inhibits the anti-tumor activity of Endostatin protein, presumably by preventing the binding of Endostatin protein to an endogenous tropomyosin protein. Just as the E37 peptide and tropomyosin have been tested in vitro and in vivo for their affect upon and relationship to Endostatin protein, the other binding peptides and binding proteins discussed herein may be tested to determine their affect upon and relationship to Endostatin and Angiostatin protein. The in vitro assays that may be used are familiar to those skilled in the art and include HUVEC and BCE proliferation assays, HUVEC wound/migration assay, endothelial cell tube forming assay, CAM assay, Matrigel invasion assay and the rat aortic assay. Specifically, the binding peptides and binding proteins that inhibit or stimulate the activity of Angiostatin protein or Endostatin protein are identified.

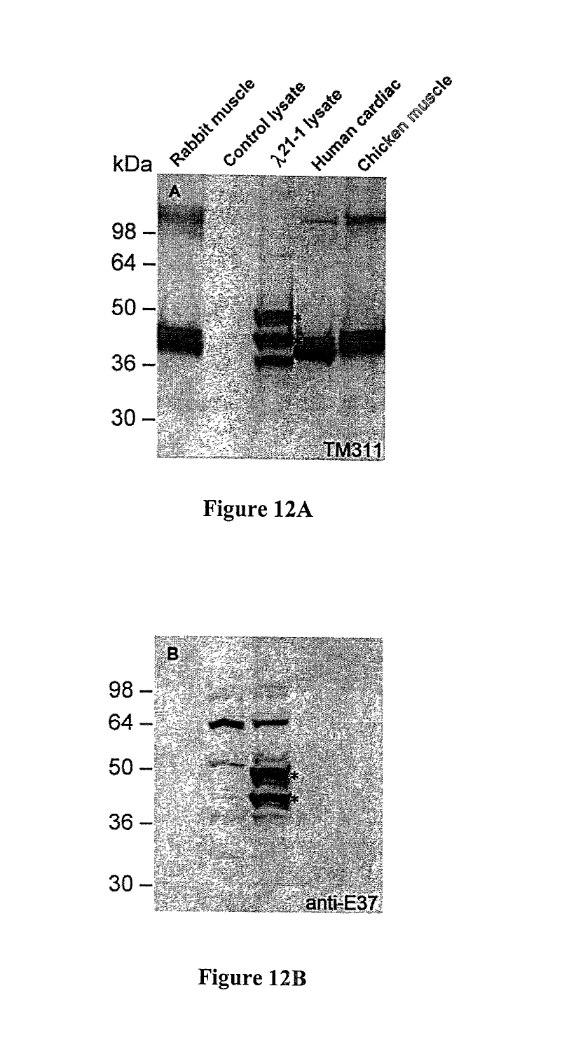

The present inventors have specifically identified human tropomyosin isoform 3 (hTM3) as an Endostatin-binding protein. Using an epitope specific antibody (FIG. 12B), they demonstrate herein that the Endostatin-binding epitope of hTM3 is not present in all tropomyosins and consequently that Endostatin-binding is presumably not a characteristic of all tropomyosin isoforms. This finding has broad significance. As previously discussed, Endostatin inhibits the growth of tumors by inhibiting endothelial cell function (O'Reilly et al. Cell 88(2), 277-85 (1997), Dhanabal et al. Cancer Res 59(1), 189-97 (1999); Sim et al. Angiogenesis 4(1) 41-51 (1999)). Systemic rhEndostatin therapy was shown to induce a 50% reduction of intratumoral blood flow while other non-tumor affected organs in the same animal were unaffected. Furthermore, other events that require angiogenesis such as wound healing remained unaffected by rhEndostatin (Berger et al. J Surg Res 91(1), 26-31 (2000); Bloch et al. FASEB J 14(15), 2373-6 (2000)). These observations suggest that the mechanism for Endostatin's effect is tightly controlled. The present inventors' finding that Endostatin may only affect specific isoforms of tropomyosin provides an explanation for how this tight control could be achieved and maintained. Though not wishing to be bound by the following theory, it is thought that specific isoforms of tropomyosin are expressed and regulated in endothelial cells during tumor angiogenesis. The inventors believe that these specific isoforms bind Endostatin preferentially leading to potent inhibition of tumor growth. It is also believed that expression of such isoforms of tropomyosin that interact with Endostatin with different affinities and dissociation constants provide the natural homeostatic balance of endothelial cell growth under normal physiological conditions.

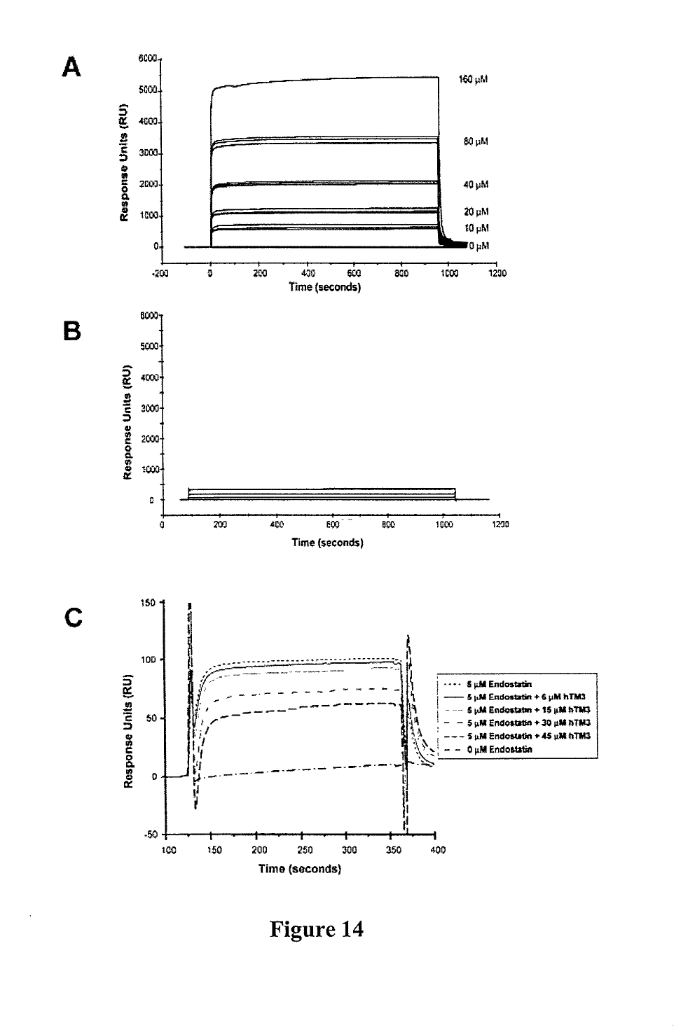

The inventors demonstrated that the binding of rhEndostatin to immobilized rhTM3 displays both rapid rates of association and dissociation and a K_{D} of ˜100 μM (FIG. 14A). Kinetic analysis of these sorts of interactions show they have relatively low affinities that are also the result of rapid dissociation rates. Such kinetics are suggestive of highly dynamic interactions. Based on the discoveries by the present inventors, the present application includes all isoforms of tropomyosin that have an affinity for Endostatin. Also included are any other naturally occurring or synthetically produced components that are involved in stabilizing the Endostatin/hTM3 interaction in vivo, wherein the presence of this molecule(s) the K_{D} of binding would be significantly lower. The present invention includes therefore, methods and compositions comprising tropomyosin isoforms such as hTM3 that have an increased affinity for antiangiogenic molecules such as Endostatin.

While pharmacokinetic analysis indicates that parenteral administration of rhEndostatin results in circulating levels in the nM range (Sim et al.), local concentrations in the tumor microenvironment may be significantly higher. Planar imaging of mammary tumor-bearing rats following i.v. injection of technetium (^{99m}Tc) labeled rhEndostatin (100 μCi/rat) showed that the tumor could be visualized from 0.5-4 hours post-injection (personal communication, David Yang). This observation suggests that Endostatin targets the tumor microenvironment and indicates significantly higher Endostatin concentrations at the tumor bed in comparison to circulating levels. Though not wishing to be bound_by the following theory, it is believed that elevated local concentrations may favor the interaction of Endostatin with hTM3 in the tumor vasculature.

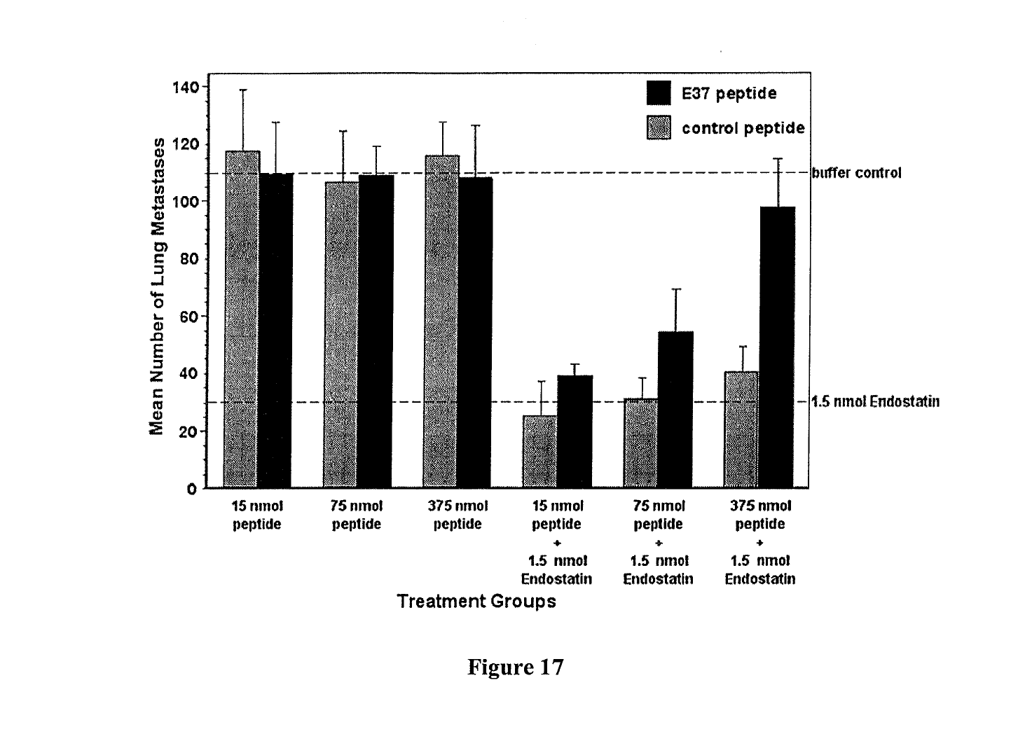

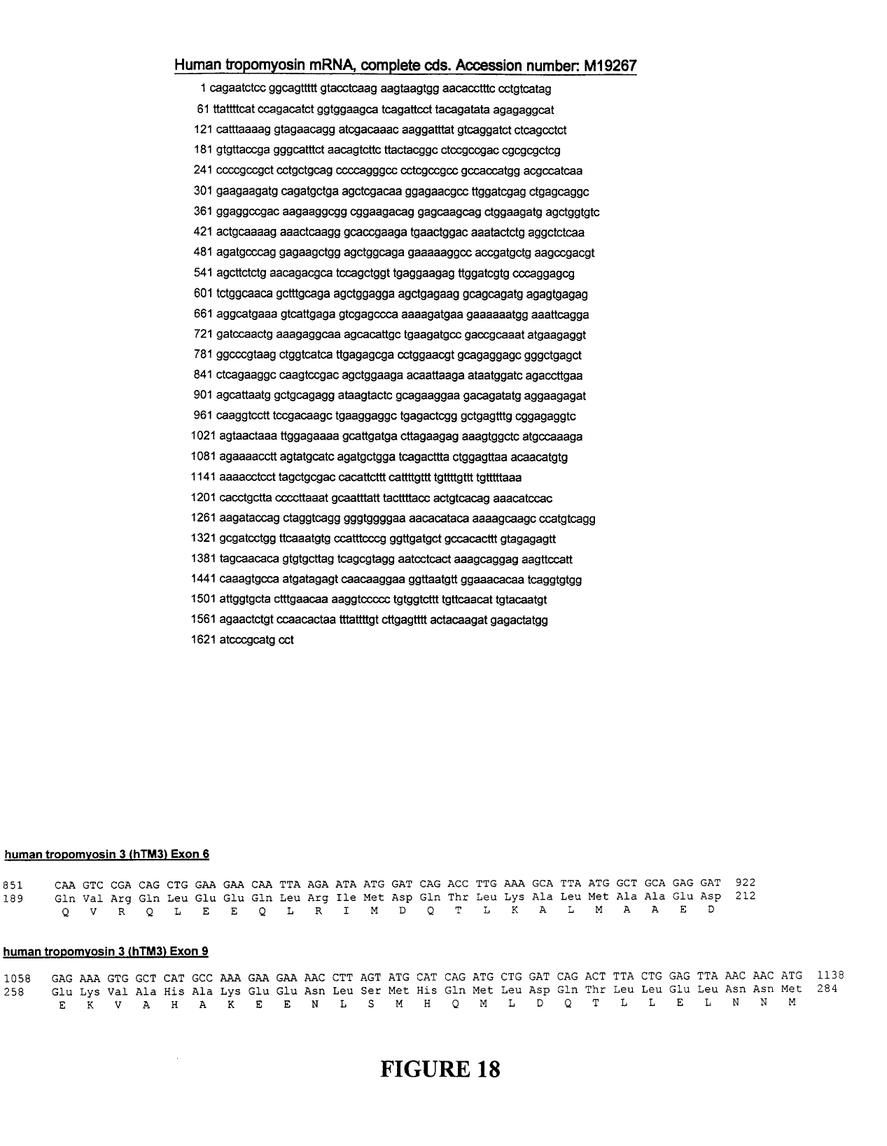

For the first time the present inventors provide evidence that links a mechanism of action to the in vivo anti-tumor activity of Endostatin. Co-injection of the E37 peptide with rhEndostatin resulted in a dose dependent inhibition of the anti-tumor activity of rhEndostatin in the B16-BL6 experimental metastasis assay (FIG. 17). It is believed that the E37 peptide mimics the Endostatin protein-binding epitope of hTM3 to compete for Endostatin binding and consequently inhibits Endostatin's anti-tumor activity.

The present inventors further provide that the anti-angiogenic effect of Endostatin is mediated via an interaction with tropomyosin-containing microfilaments. Rearrangement of the actin cytoskeleton has been reported to be characteristic of, and sufficient to induce endothelial cell apoptosis (DeMeester et al. Surgery 124(2), 362-71 (1998)). Taken together, rhEndostatin's ability to induce endothelial cell apoptosis, and the inventors' observations that rhEndostatin binds tropomyosin-containing mircofilaments, suggests that Endostatin disrupts microfilament function to initiate apoptosis. Further observations consistent with Endostatin exerting an effect upon the actin cytoskeleton come from an endothelial cell monolayer-wound assay (Sim et al.; Sato et al. J Cell Biol 107(3) 1199-205 (1988)). In this assay, cell migration at the wound edge was correlated with increased incorporation of monomeric actin into filaments and a reorganization of the actin cytoskeleton (Moldovan et al. Circ Res 86(5) 549-57 (2000)). Based on the inventors novel findings of the interaction between Endostatin and tropomyosin, and tropomyosin and microfilaments, it is demonstrated for the first time that Endostatin binding to microfilaments may inhibit the cytoskeletal reorganization required for cell migration, leading to an inhibition of migration.

While the mechanism by which the Endostatin and hTM3 interaction may induce endothelial cell apoptosis is not completely defined, tropomyosin and the cytoskeleton have critical roles in cell survival. Ingber and co-workers have demonstrated that actin-based microfilaments play a crucial role in cell shape-regulated determination of cell fate (Ingber et al. Cell 58(5), 803-5 (1989); Ingber et al. Proc Natl Acad Sci_USA 87(9), 3579-83 (1990); Huang et al. Mol Biol Cell 9(11), 3179-93 (1998); Dike et al. In Vitro Cell Dev Biol Anim 35(8), 441-8 (1999)) and that while growth factors and integrin signaling are required for endothelial cell growth, they are not sufficient (Ingber et al. J Cell Biol 110(5), 1803-11 (1990)). Progression through the cell cycle is inhibited following disruption of the cytoskeleton or by release of cytoplasmic tension, implicating the actin-based cytoskeleton and its ability to generate tension against integrin-ECM contacts in the regulation of endothelial cell cycle progression and cell fate (Huang et al. Mol Biol Cell 9(11), 3179-93 (1998); Pourati et al. Am J Physiol 274(f Pt 1), C1283-9 (1998)) The novel findings of present inventors demonstrate herein that Endostatin, binds cytoskeletal tropomyosin leading to a release of cytoskeletal tension and subsequent induction of cell apoptosis.

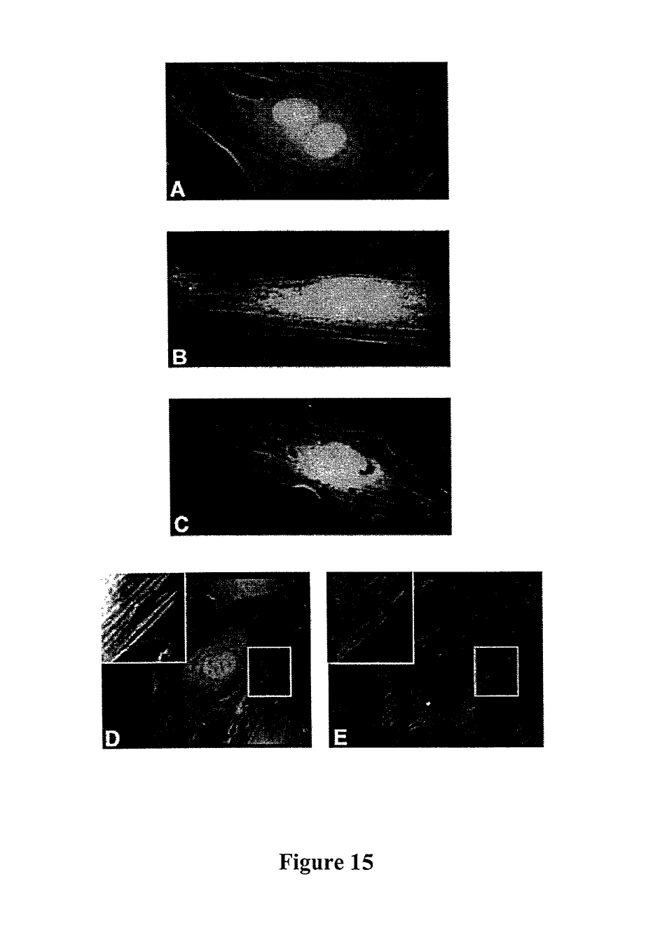

Endothelial cells rapidly internalize Endostatin (Dixelius et al. Blood 95(11), 3403-11 (2000)), and the inventors show here for the first time that rhEndostatin and tropomyosin surprisingly co-localize to the microfilaments of formalin fixed human endothelial cells (FIG. 15). The biological relevance of the tropomyosin/rhEndostatin interaction to the antiangiogenic activity of Endostatin was demonstrated by blocking anti-tumor activity of rhEndostatin with a peptide mimotope of tropomyosin. Though no wishing to be bound by the following theory, it is postulated therefore that the antiangiogenic activity of Endostatin may result, at least in part, from internalization of Endostatin by endothelial cells and its subsequent interaction with tropomyosin; leading to a disruption of microfilament integrity, inhibition of cell motility and induction of apoptosis and ultimately tumor growth inhibition.

As a result of the inventors findings, also included herein are_antiangiogenic actin-binding molecules. Though not wishing to be bound by the following theory, it is believed that since actin polymerization is affected by Endostatin it could possibly mean that Endostatin is binding to the actin component of the microfilaments, in addition to or in association with tropomyosin. A study of actin polymerization in the presence of Endostatin using a light scatter assay demonstrates that Endostatin may be binding G-actin (globular, G-actin polymerizes to form F-actin (filamentous)) and that the interacting G-actin/Endostatin molecules form nucleation complexes that shorten the lag phase and facilitate actin polymerization. Accordingly, molecules such as Endostatin that have an effect on actin polymerization will have an effect on microfilaments resulting in anti-angiogenic activity through modification of cell motility, and induction of cell apoptosis. Preferred embodiments of the present invention include compositions comprising proteins and chemicals that bind tropomyosin isoforms, such as hTM3. In certain embodiments, such compositions may be characterized as antiangiogenic actin-binding molecules.

Identification and Analysis of Binding Peptides

The biological activity of the binding peptides and binding proteins can also be tested in vivo. The binding peptides and binding proteins are pre-incubated with their target angiogenesis-related protein (Angiostatin protein or Endostatin protein) prior to being used in either the B16B16 metastasis assay or the Lewis Lung Carcinoma primary tumor or metastasis assays. In such experiments a comparison is made between the activity of the Angiostatin or Endostatin protein and the Angiostatin or Endostatin protein pre-bound with the binding peptide or binding protein. If the binding peptide (or protein)/target protein interactions mimic important biological interactions involved in the activity of Angiostatin protein or Endostatin protein, then it would be expected that the anti-angiogenic activity of the target protein would be neutralized by the binding of the binding peptide or binding protein.

In addition, phage display may be used to identify other binding peptides and proteins for better understanding, and ultimately modifying, the role of angiogenesis-related proteins in angiogenesis, and in the manifestation of angiogenesis-related disease. For example, the binding peptides may be used to identify proteins that interact with, and/or regulate (either positively or negatively), the activity of angiogenesis-related proteins such as Angiostatin protein or Endostatin protein. In addition, such binding peptides may also be used to identify other proteins and molecules involved in the transport of angiogenesis-related proteins, and substrates through which angiogenesis-related proteins exert their activities.

In addition to encompassing Angiostatin and Endostatin binding peptides and proteins, the present invention further includes tropomyosin binding compounds and methods of inhibiting angiogenesis in an individual comprising administering to the individual an angiogenesis inhibiting amount of a tropomyosin binding compound. As used herein, the term “tropomyosin binding compound” refers to a protein, chemical, or lipid that specifically binds to a tropomyosin protein and thereby inhibits angiogenesis. In one embodiment of the present invention, the tropomyosin protein is a non-muscle cell tropomyosin protein. In a preferred embodiment of the present invention, the non-muscle cell tropomyosin protein is a fibroblast tropomyosin. In another embodiment of the present invention, the tropomyosin protein comprises a contiguous span of amino acids having substantial homology to amino acids 189-212 of fibroblast tropomyosin (SEQ ID NO:116), amino acids 258-284 of fibroblast tropomyosin (SEQ ID NO:117), or a combination thereof.

Tropomyosin binding compounds are identified and isolated by methods known to those of skill in the art. Examples of methods that may be used to identify tropomyosin binding compounds are the yeast-2-hybrid screening, phage display affinity chromatography, expression cloning and Biacore systems. Biacore systems are used to identify chemical mimetics of an Endostatin protein as these systems enable direct detection and monitoring of biomolecular binding events in real time without labeling and often without purification of the substances involved. (Biacore, Rapsagatan 7, SE 754 50 Uppsala.)

In particular, the yeast-2-hybrid screening approach utilizes transcription activation to detect protein-protein interactions. Many transcription factors can be separated into two domains, a DNA binding domain and a transcriptional activation domain that are inactive when separated. When the two domains are brought into ‘close proximity’ their functional transcriptional activation activity is recreated. In the present invention, an Endostatin protein, an Angiostatin protein, or a tropomyosin protein (the protein of interest) is fused to a transcription factor DNA binding domain and cDNAs from a cDNA library are fused to a sequence encoding a transcriptional activation domain. A yeast strain which has been transformed with the cDNA encoding the protein of interest fused to a transcription factor DNA binding domain, is transformed with the transcriptional activation domain/cDNA fusion library. Any cDNA which codes a protein that binds to the protein of interest will allow the formation of a functional hybrid transcriptional activator (as the DNA binding and transcriptional activation domains are now in ‘close proximity’) leading to the expression of a reporter gene that results in cell survival. The cDNA coding the binding protein is then isolated and the protein that it encodes identified.

The present invention still further includes methods of inhibiting angiogenesis in an individual comprising administering to the individual an angiogenesis inhibiting amount of an actin cytoskeleton disrupting compound. In one embodiment of the present invention, the actin cytoskeleton disrupting compound is a tropomyosin binding compound. Actin disrupting compounds as defined herein are identified and isolated by methods known to those of skill in the art. As defined herein, an actin cytoskeleton disrupting compound is a protein, chemical or lipid that disrupts the actin cytoskeleton of an endothelial cell and thereby inhibits angiogenesis. Disruption of the actin cytoskeleton refers to the rearrangement of the actin cytoskeleton and/or a change in the tension of the actin cytoskeleton. Rearrangement of the actin cytoskeleton includes disaggregation of the actin filaments as induced, for example, by Cytochalasin D. Rearrangement of the actin cytoskeleton also includes the subtle rearrangements of the cytoskeleton that occur during stress-induced apoptosis. A change in the tension of the actin cytoskeleton is important because the integrity of the cytoskeleton and its ability to generate tension against a resisting substrate through integrin-ECM interactions may regulate cell shape. Altering cell geometry of cell spreading can switch endothelial cells among three major genetic programs that determine angiogenesis, growth, apoptosis and differentiation. Therefore, in one embodiment, the actin disrupting compound releases the tension of the actin cytoskeleton.

The present invention further encompasses the use of Endostatin binding peptides and proteins and Angiostatin binding peptides and proteins, such as receptors, for the detection of Endostatin and Angiostatin, respectively, in bodily fluids and tissues for the purpose of diagnosis or prognosis of angiogenesis-related diseases. As used herein, the term “angiogenesis-related disease” refers to diseases and conditions including, but not limited to, hemangioma, solid tumors, blood borne tumors, leukemia, metastasis, telangiectasia, psoriasis, scleroderma, pyogenic granuloma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, diabetic neovascularization, macular degeneration, wound healing, peptic ulcer, Helicobacter related diseases, fractures, keloids, vasculogenesis, hematopoiesis, ovulation, menstruation, placentation, and cat scratch fever. The present invention also includes methods of treating or preventing angiogenesis-related diseases including, but not limited to, arthritis and tumors by using Endostatin binding peptides and proteins and Angiostatin binding peptides and proteins for altering the expression and activity of Endostatin protein and Angiostatin protein, respectively.

The present invention further encompasses compositions comprising, vectors containing nucleotide sequences encoding the binding peptides and binding proteins described herein, wherein the vector is capable of expressing such binding peptides and binding proteins when present in a cell. The present invention also includes a composition comprising a cell containing such a vector and a method comprising, implanting into a human or non-human animal, a cell containing such a vector. Nucleotide sequences of preferred binding peptides are provided as SEQ ID NOS:43-80.

The present invention also encompasses gene therapy whereby genes encoding binding peptides and binding proteins, are regulated in an individual. Various methods of transferring or delivering DNA to cells for expression of the gene product protein, otherwise referred to as gene therapy, are disclosed in Gene Transfer into Mammalian Somatic Cells in vivo, Yang, N., Crit. Rev. Biotechn. 12(4): 335-356 (1992). Gene therapy encompasses incorporation of DNA sequences into somatic cells or germ line cells for use in either ex vivo or in vivo therapy. Gene therapy functions to replace genes, augment normal or abnormal gene function, and to combat infectious diseases and other pathologies.

Methods for treating medical problems with gene therapy include therapeutic strategies such as identifying the defective gene and then adding a functional gene to either replace the function of the defective gene or to augment a slightly functional gene; or prophylactic strategies, such as adding a gene for the product protein that will treat the condition or that will make the tissue or organ more susceptible to a treatment regimen. As an example of a prophylactic strategy, a gene for a peptide or protein comprising for example, laminin protein that binds to Angiostatin protein, and/or tropomyosin protein that binds Endostatin protein, may be placed in an individual to modify the occurrence of angiogenesis.

Many protocols for the transfer of binding peptide and binding protein DNA or binding peptide and binding protein regulatory sequences are envisioned in this invention. Transfection of promoter sequences, other than one normally found specifically associated with a binding peptide and binding protein, or other sequences that would increase production of binding peptide and binding proteins are envisioned as methods of gene therapy. Such “genetic switches” could be used to activate a binding peptide and binding protein in cells not normally expressing binding peptide and binding protein.

Gene transfer methods for gene therapy fall into three broad categories: (1) chemical (lipid-based carriers, or other non-viral vectors), (2) biological (virus-derived vector and receptor uptake), and (3) physical (electroporation, direct gene transfer and particle bombardment). Gene therapy methodologies can also be described by delivery site. Fundamental ways to deliver genes include ex vivo gene transfer, in vitro gene transfer, and in vivo gene transfer. In ex vivo gene transfer, cells are taken from the individual and grown in cell culture. The DNA is transfected into the cells, the transfected cells are expanded in number and then re-implanted in the individual. The present invention encompasses the removal of endothelial cells from an individual, transfection of DNA encoding a binding peptide and binding protein, or regulatory sequence thereof, and re-introduction of the transfected endothelial cells into the individual. In in vitro gene transfer, transformed cells, such as endothelial cells, growing in culture are introduced into the individual. The transformed cells are not taken from the individual who will receive the gene therapy. In vivo gene transfer involves introducing the DNA into the cells of the individual when the cells are within the individual. Methods include using a noninfectious virus to introduce a gene into an individual or injecting naked DNA into a site in the individual whereby DNA is taken up by a percentage of cells in which the gene product protein is expressed. In the present invention, DNA encoding a binding peptide and binding protein can be introduced into the endothelial cells lining the blood vessels, thereby inhibiting angiogenesis. In a preferred embodiment, the DNA encoding a binding peptide or binding protein is introduced into endothelial cells lining the blood vessels in close proximity to or within a tumor.

Chemical methods of gene therapy may involve a lipid based compound, not necessarily a liposome, used to ferry the DNA across the cell membrane. Lipofectins or cytofectins, lipid-based positive ions that bind to negatively charged DNA, make a complex that can cross the cell membrane and provide the DNA into the interior of the cell. Liposome/DNA complexes may be directly injected intravenously into the individual. It is believed that the liposome/DNA complexes are concentrated in the liver where they deliver the DNA to macrophages and Kupffer cells. These cells are long lived and thus provide long term expression of the delivered DNA. Additionally, vectors or the “naked” DNA of the gene may be directly injected into the desired organ, tissue or tumor for targeted delivery of the therapeutic DNA. Other DNA carrier systems include the asialoglycoprotein/polylysine conjugate system for carrying DNA to hepatocytes for in vivo gene transfer and DNA coupled to nuclear proteins in specifically engineered vesicle complexes that are carried directly into the nucleus.

Biological methods used in gene therapy techniques may involve receptor-based endocytosis, or receptor-based phagocytosis, which involve binding a specific ligand to a cell surface receptor and enveloping and transporting the ligand across the cell membrane. Specifically, a ligand/gene complex is created and injected into the blood stream. Target cells having a receptor for the ligand will specifically bind the ligand and transport the ligand-DNA complex into the cell. Additional biological methods employ viral vectors to insert genes into cells. For example, altered retrovirus vectors have been used in ex vivo methods to introduce genes into peripheral and tumor-infiltrating lymphocytes, hepatocytes, epidermal cells, myocytes, and other somatic cells. These altered cells are then introduced into the individual.

Viral vectors have also been used to insert genes into cells using in vivo protocols. To accomplish tissue-specific expression of foreign genes, cis-acting regulatory elements or promoters that are known to be tissue specific can be used. Alternatively, tissue-specific expression can be achieved using in situ delivery of DNA or viral vectors to specific anatomical sites in vivo. For example, gene transfer to blood vessels in vivo has been achieved by implanting in vitro transduced endothelial cells in chosen sites on arterial walls. Surrounding cells were infected by the virus and therefore also expressed the gene product. A viral vector can be delivered directly to the in vivo site, by a catheter for example, thus allowing only certain areas to be infected by the virus. In vivo gene transfer using retrovirus vectors has also been demonstrated in mammary tissue and hepatic tissue by injection of the altered virus into blood vessels leading to the organs.

Viral vectors that have been used for gene therapy protocols include, but are not limited to, retroviruses such as murine leukemia retroviruses, RNA viruses such as poliovirus or Sindbis virus, adenovirus, adeno-associated virus, herpes viruses, SV40, vaccinia and other DNA viruses. Replication-defective murine retroviral vectors are the most widely utilized gene transfer vectors. Fundamental advantages of retroviral vectors for gene transfer include efficient infection and gene expression in most cell types, precise single copy vector integration into target cell chromosomal DNA, and ease of manipulation of the retroviral genome. The adenovirus is capable of transducing novel genetic sequences into target cells in vivo. Adenoviral-based vectors express gene product proteins at high levels and have high efficiencies of infectivity, even with low titers of virus. Additionally, the virus is fully infective as a cell free virion so injection of expression cell lines is not necessary. Another potential advantage to adenoviral vectors is the ability to achieve long term expression of heterologous genes in vivo.

Mechanical methods of DNA delivery include direct injection of DNA, such as microinjection of DNA into germ or somatic cells, pneumatically delivered DNA-coated particles, such as the gold particles used in a “gene gun,” inorganic chemical approaches such as calcium phosphate transfection and electroporation. It has been found that injecting plasmid DNA into muscle cells yields high percentage of the cells that are transfected and have sustained expression of marker genes. The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be re-injected periodically to maintain the gene product level without mutations occurring in the genomes of the recipient cells. Non-integration of exogenous DNAs may allow for the presence of several different exogenous DNA constructs within one cell with all of the constructs expressing various gene products.

Both particle-mediated gene transfer methods and electroporation can be used in in vitro systems, or with ex vivo or in vivo techniques to introduce DNA into cells, tissues or organs. With regard to particle-mediated gene transfer, a particle bombardment device, or “gene gun,” is used that generates a motive force to accelerate DNA-coated high density particles (such as gold or tungsten). These particles penetrate the target organs, tissues or cells. Electroporation mediated gene transfer comprises the use of a brief electric impulse with a given field strength that is used to increase the permeability of a membrane in such a way that DNA molecules can penetrate into the cells.

The gene therapy protocol for transfecting DNA encoding binding peptide and binding proteins into a individual may either be through integration of the binding peptide and binding protein DNA into the genome of the cells, into minichromosomes or as a separate replicating or non-replicating DNA construct in the cytoplasm or nucleoplasm of the cell. Binding peptide and binding protein expression may continue for a long-period of time or the DNA may be re-injected periodically to maintain a desired level of the binding peptide and binding protein in serum or in a cell, tissue or organ.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

The following materials and methods were used in conducting the experiments for the present invention.

Screening of the Phage-display and cDNA Libraries

The Ph.D.-C7C, a disulfide constrained 7-mer phage-display library (New England BioLabs, Beverly, Mass.), was screened as recommended by the manufacturer. Following a third round of amplification individual phage were isolated and the peptide sequences deduced by DNA sequencing.

Peptide Synthesis and Generation of Anti-peptide Sera

The E37 (CTHWWHKRCGGGS) and control (CSNSDKPKCGGGS (SEQ ID NO:118) peptides were synthesized, cyclized at high dilution, and purified to at least 95% purity by high-performance liquid chromatography (Infinity Biotech Research and Resource, Aston, Pennsylvania). The E37 peptide was coupled to Hemocyanin from Keyhole Limpets (Sigma, St. Louis, Miss.) in the presence of glutaraldehyde and used to immunize New Zealand White rabbits.

cDNA Library Screening

The Lambda ZAP® II (Stratagene, La Jolla, Calif.) bFGF stimulated HUVEC cDNA library was constructed by directional cloning of the oligo (dT) primed cDNA into EcoRI and XhoI cloning sites. 1.4×10⁶ recombinant phage were screened for immunoreactivity to E37 peptide anti-sera.

Native and Recombinant Proteins

Plasmid pλ21-1 was excised as a pBluescript phagemid (as recommended by Stratagene) from a recombinant phage isolated from the HUVEC cDNA library based on its recognition by E37 anti-sera. DNA sequencing of the cDNA insert of pλ21-1 showed it contained the complete hTM3 coding sequence fused in frame downstream of the Lac Z gene. E. coli DH5a cells were transformed with pλ21-1 and pBluescript plasmids and crude bacterial lysates were prepared. Human cardiac tropomyosin and muscle tropomyosins were purchased from Trichem Resources (West Chester, Pa.) and Sigma (St. Louis, Miss.) respectively.

The cDNA encoding hTM3 cDNA was amplified by PCR using forward primer #596 (5′-ATGCCATATGGACGCCATCAAGAAG-3′ (SEQ ID NO:120)) and reverse primer #597 (5′-ATGCAAGCTTTCACATGTTGTTTAACTCCAG-3′ (SEQ ID NO: 121) and cloned into pET-21a(+) (Novagen, Madison, Wis.). BL21 (DE3) cells were transformed with the p6T plasmid and grown in LB containing 50 μg/ml carbenicillin (Novagen, Madison, Wis.). The cells were induced with 5 mM IPTG, harvested by centrifugation, resuspended in 1M Tris-HCl pH8, 1M NaCl and heated 45 minutes at 90° C. The lysate was cooled to room temperature, centrifuged and the supernatant loaded onto a Sulfopropyl Sepharose Fast Flow column (SP-FF; Amersham Pharmacia, Piscataway, New Jersey) which had been equilibrated in a 50 mM phosphate buffer pH 7.5. The proteins were eluted with a linear NaCl gradient, the recombinant (r) hTM3 fractions were acidified to a pH of 2 with TFA, loaded onto a reverse phase C4 column (Vydac Inc., Hesperia, Calif.) and eluted with an acetonitrile/TFA gradient. The fractions containing hTM3 were concentrated and dialyzed against PBS. rhAngiostatin K1-3 (Liang et al., unpublished data) and rhEndostatin were produced in P. pastoris and purified to homogeneity.

ELISA

Plates were coated with 5 μg/ml of BSA or rhEndostatin and blocked. Dilutions of λ21-1 or control lysate were added and incubated for 1 hour at 37° C., followed by incubation with 1:500 diluted anti-tropomyosin TM311 ascites fluid (Sigma, St. Louis, Miss.). Following incubation in 1:5000 diluted anti-mouse IgG (H+L) conjugated to alkaline phosphatase (Promega, Madison, Wis.) wells were developed using Blue Phos Phosphatase Substrate (Kirkegaard and Perry, Gaithersburg, Md.) and the OD₆₃₅ measured.

Surface Plasmon Resonance (BIAcore)

Interactions between purified soluble rhEndostatin and rhTM3 were evaluated by surface plasmon resonance using the BIAcore 3000 (BiaCore, Piscataway, N.J.). The purified rhTM3 was immobilized on the flow cell of a CM-5 BIAcore biosensor chip. The running buffer was 0.01 M HEPES, pH 7.4, 0.15M NaCl and 0.005% polysorbate 20 (v/v). The competition experiments involving soluble rhTM3 were performed using PBS pH 7.4 as running buffer. All measurements were performed at 25° C. To correct for differences in bulk refraction all protein preparations were passed over an activated and blocked flow cell to which no protein had been coupled. Effects due to bulk shift were subtracted from experimental data.

Fluorescent Staining of Endothelial Cells

To detect the binding of Alexa-labeled rhEndostatin 1×10⁴ HUVEC, HMVEC and HAECs were plated on 1.5% gelatin coated Nalge Nunc Lab Tek II chamber slides (Naperville, Ill.) and fixed in 10% neutral buffered formalin followed by a methanol wash. The chamber slides were incubated in PBS/1% calf serum containing 40 μg/ml (2 μM) of Alexa 488-labeled rhEndostatin (Narum et al., manuscript in preparation) for 1 hr at room temperature. The slides were washed in PBS, coverslips mounted using fluorescent mounting media (Kirkegaard and Perry Laboratories, Gaithersburg, Md.) and cells photographed under Alexa 488 excitation wavelength. Co-localization studies were performed as above except cells were incubated in PBS/1% calf serum containing 1:200 mouse anti-tropomyosin TM311 (Sigma, St. Louis, Miss.) and 40 μg/ml of Alexa 488-labeled rhEndostatin followed by incubation with goat anti-mouse IgG (H+L) conjugated to Alexa 594 (Molecular Probes, Eugene, Oregon). The same fields were photographed under Alexa 488 and Alexa 594 wavelengths. Competition experiments were done by including 100 μM of E37 or control peptide in the 40 μg/ml (2 μM)of Alexa-rhEndostatin solution used to stain the cell.

In vivo Studies

C57BL/6J mice (n=8) were injected intravenously (i.v.) with 5×10⁴ B16-BL6 cells/mouse. Three days later treatment commenced. 100 μl of citrate-phosphate buffer (66 mM Sodium Phosphate, 17 mM Citric acid, 59 mM NaCl pH 6.2) containing 1.5 nmol of rhEndostatin and 100 μl of PBS containing either 0, 15, 75 or 375 nmol of control or E37 peptide were mixed and injected subcutaneously (s.c.) daily for 11 days. All mice were then sacrificed, their lungs removed and the number of surface metastases counted. Results were analyzed for statistical significance using the 2-tailed Student's t-test.

To determine the pharmacokinetics, C57BL/6J mice (3 per time point) were dosed s.c. with 1.5 nmol of rhEndostatin alone or in combination with 375 nmol of either E37 or control peptide. Sera was collected 0.17, 0.5, 1 and 2 hours following s.c. administration. Serum drug levels were determined using an Endostatin EIA kit (Cytlmmune Sciences, College Park, Md.) according to the manufacture's instructions. Pharmacokinetic analyses of the serum concentration versus time data were performed by nonlinear least square minimization using RSTRIP (Version 4.03, MicroMath Scientific Software, Salt Lake City, Ut.).

### EXAMPLE 1

### Identification of Angiogenesis-related Binding Peptides

A “phage-display library” is a protein expression library, constructed in a vector that expresses a collection of cloned protein sequences as fusions with a phage coat protein. Thus, in the context of the present invention, single-chain recombinant proteins having the potential ability to bind angiogenesis-related proteins, are expressed as fusion proteins on the exterior of the phage particle. This “placement” advantageously allows contact and binding between the recombinant binding protein and an immobilized protein such as Angiostatin protein or Endostatin protein. Phage that bind an angiogenesis-related protein can be recovered; individual phage can then be cloned and the peptide expressed by cloned phage can be determined. Phage clones expressing binding peptides specific for angiogenesis-related proteins can be substantially enriched by serial rounds of phage binding to the immobilized protein and amplification by growth in bacterial host cells.

Methods for preparing libraries containing diverse populations of various types of molecules such as peptides, polypeptides, protein, and fragments thereof are well known in the art and are commercially available (see, for example, Ecker and Crooke, Biotechnology 13:351-360 (1995), and the references cited therein).

Where a molecule is a peptide, protein or fragment thereof, the molecule can be produced in vitro directly or can be expressed from a nucleic acid, which is produced in vitro. Methods of synthetic peptide and nucleic acid chemistry are well known to those skilled in the art.

A library of molecules can also be produced, for example, by constructing a cDNA expression library from mRNA collected from a cell, tissue, organ or organism of interest. Methods for producing such libraries are well known in the art (see, for example, Sambrook et al. Molecular Cloning: A laboratory manual (Cold Spring Harbor Laboratory Press 1989, latest edition)). Preferably, the peptide encoded by the cDNA is expressed on the surface of a cell or a virus containing the cDNA. For example, cDNA can be cloned into a phage vector wherein, upon expression, the encoded peptide is expressed as a fusion protein on the surface of the phage.

Phage display technology was used to identify peptide and protein sequences that bind Angiostatin protein or Endostatin protein, and also to identify peptides that mimic the effector binding sites of effector molecules that interact with Angiostatin protein and Endostatin protein.

All cloning procedures employed in the development of the present invention were carried out according to standard laboratory practice.

Phage Display Libraries

The Ph.D.-7 phage display library (New England BioLabs Cat #8100), a combinatorial library consisting of random peptide 7-mers, was screened for 7 amino acid binding peptides. The Ph.D.-7 phage display library consists of linear 7-mer peptides fused to the pIII coat protein of M13 via a Gly-Gly-Gly-Ser flexible linker. The library contains 2.8×10⁹ independent clones. The Ph.D. library is useful for identifying targets requiring binding elements concentrated in a short stretch of amino acids.

The Ph.D.-C7C library (New England BioLabs Cat # 8120) is a combinatorial library consisting of random peptide 7-mers flanked by cysteine residues that under non-denaturing conditions are disulfide bonded resulting in the display of cyclized peptides. In non-reducing conditions, the cysteines form a disulfide bond resulting in each peptide being constrained in a disulfide loop. The library contains 3.7×10⁹ independent clones that, like the Ph.D.-7 library, result in peptides fused to the pIII coat protein of M13 via a Gly-Gly-Gly-Ser flexible linker. Constrained libraries are useful in the identification of structural epitopes. The Ph.D.-C7C library was screened for the binding of 7 amino acid cyclized binding peptides.

Phage Selection

The phage display libraries were screened following the manufacturers' recommendations. Approximately 100 μg/ml of recombinant human Angiostatin protein, K1-4 protein, or recombinant human Endostatin protein were coated overnight onto 6-mm tissue culture plates at 4° C. in a humidified container. In the first round of panning, approximately 2×10¹¹ phage were incubated on the protein coated plate for 60 minutes at room temperature while rocking gently. The plates were washed six times using TBS (50 mM Tris-HCl (pH 7.5), 150 mM NaCl) containing 0.1% Tween 20. The binding phage were collected and amplified following elution using 100 μg/ml of the target protein. Secondary and tertiary pannings were performed as for the primary screen except the TBS washing buffer contained 0.5% Tween 20.

Sequencing of Angiostatin Binding Peptides

Following the tertiary screening, 10-12 individual colonies of phage-infected bacteria were picked at random, the phage DNA was isolated and subjected to automated dideoxy sequencing. The sequence of the displayed peptides were deduced from the DNA sequence. The binding peptides presented below in Tables 1-4 were identified as linear or cyclized peptides that bind Angiostatin protein or Endostatin protein. The nucleic acid sequences corresponding to the amino acid sequences shown in Tables 1-4 are provided as SEQ ID NOS:43-80.

TABLE 1
Linear Angiostatin Protein Binding Peptides

| Peptide | Amino Acid Sequence | SEQ ID NO: |
|---|---|---|
| A1 | E R V N D D T G G G S | SEQ ID NO:3 |
| A3 | D R S G A I K G G G S | SEQ ID NO:4 |
| A7 | L D R A N V F G G G S | SEQ ID NO:5 |
| A9 | S P L G G S E G G G S | SEQ ID NO:6 |
| A10 | H A I Y P R H G G G S | SEQ ID NO:7 |

TABLE 2
Cyclized Angiostatin Protein Binding Peptides

| Peptide | Amino Acid Sequence | SEQ ID NO: |
|---|---|---|
| A25 | C W S Y E W S K C G G G S | SEQ ID NO:8 |
| A31 | C W S L E Q S K C G G G S | SEQ ID NO:9 |
| A35 | C W S L E W Q K C G G G S | SEQ ID NO:10 |
| A28 | C W S L E T T K C G G G S | SEQ ID NO:11 |
| A33 | C W S L E H Q K C G G G S | SEQ ID NO:12 |
| A34 | C W S L E I L K C G G G S | SEQ ID NO:13 |
| A30 | C W T L E S T K C G G G S | SEQ ID NO:14 |
| A32 | C G D M S D R P C G G G S | SEQ ID NO:15 |

TABLE 3
Linear Endostatin Protein Binding Peptides

| Peptide | Amino Acid Sequence | SEQ ID NO: |
|---|---|---|
| E15 | H K R P R N N G G G S | SEQ ID NO:16 |
| E12 | T K H R A G R G G G S | SEQ ID NO:17 |
| E13 | W H R S V W K G G G S | SEQ ID NO:18 |
| E14 | S P Q P F E E G G G S | SEQ ID NO:19 |
| E16 | F T E P T H K G G G S | SEQ ID NO:20 |
| E17 | K D Y A L P P G G D S | SEQ ID NO:21 |
| E18 | S K I A P I M G G G S | SEQ ID NO:22 |
| E20 | W R Q T R K D G G G S | SEQ ID NO:23 |
| E22 | G K P M P P M G G G S | SEQ ID NO:24 |
| E37 | C T H W W H K R C G G G S | SEQ ID NO:25 |

TABLE 4
Cyclized Endostatin Protein Binding Peptides

| Peptide | Amino Acid Sequence | SEQ ID NO: |
|---|---|---|
| E41 | C S L T P H R Q C G G G S | SEQ ID NO:26 |
| E45 | C E K E K P M T C G G G S | SEQ ID NO:27 |
| E48 | C A P P G L A R C G G G S | SEQ ID NO:28 |

### EXAMPLE 2

Identification of Binding Peptides that Specifically Bind Angiostatin protein over Plasminogen

Both the linear (Ph.D.-7) and constrained (Ph.D.-C7C) libraries as described in Example 1 were screened for Angiostatin protein specific binding clones: phage that specifically bind to Angiostatin protein as opposed to plasminogen. Four rounds of screening were performed on both libraries as follows:

Round 1

2×10¹¹ phage bound to Angiostatin protein coated plate

plated washed 10 times

eluted phage with 100 μg/ml plasminogen

plated washed 10 times

eluted phage with 100 μg/ml Angiostatin protein K1-4

amplified the Angiostatin protein eluted phage

Round 2

2×10¹¹ amplified primary screen Angiostatin protein eluted phage bound to Angiostatin protein coated plate

plated washed 10 times

eluted phage with 100 μg/ml plasminogen plated washed 10 times eluted phage with 100 μg/ml Angiostatin protein K1-4

amplified the Angiostatin protein eluted phage

Round 3

2×10¹¹ amplified secondary screen Angiostatin protein eluted phage bound to Angiostatin protein coated plate

plated washed 10 times

eluted phage with 200 μg/ml lys-plasminogen

plated washed 10 times

eluted phage with 100 μg/ml Angiostatin protein K1-4

amplified the Angiostatin protein eluted phage

Round 4

2×10¹¹ amplified tertiary screen Angiostatin protein eluted phage bound to Angiostatin protein coated plate

plated washed 10 times

eluted phage with 200 μg/ml lys-plasminogen

plated washed 10 times

eluted phage with 100 μg/ml Angiostatin protein K1-4

Eluted phage were titrated and plated at approximately 75 pfu per plate. Twenty four individual plaques were picked from the linear (clones PAL 49 to PAL-72) and the constrained (clones PAC-73 to PAC-96) libraries and the phage were amplified. Untitrated phage were assayed for their ability to bind to Angiostatin protein and plasminogen in an ELISA assay. Four-fold serial dilutions of phage ranging from 1:4 to 1:65 536 were tested. As shown in Tables 5 and 6, several phage were identified as specifically binding to Angiostatin protein over plasminogen protein. These phage were selected for further analysis.

TABLE 5
Linear Angiostatin Binding Peptides that
Bind Preferentially to Angiostatin protein
over Plasminogen

| Peptide | Amino Acid Sequence | SEQ ID NO: |
|---|---|---|
| PAL-49 | K C C Y Y A K G G G S | SEQ ID NO:31 |
| PAL-51 | K C C Y P S A G G G S | SEQ ID NO:32 |
| PAL-54 | R Q P P H L H G R G S | SEQ ID NO:33 |
| PAL-56 | H K Y I S A T G G G S | SEQ ID NO:34 |
| PAL-66 | G T L Q V L S G G G S | SEQ ID NO:35 |
| PAL-69 | K C C Y S V G G G G S | SEQ ID NO:36 |
| PAL-70 | M S Y Q W S H G G G S | SEQ ID NO:37 |

TABLE 6
Cyclized Angiostatin Binding Peptides that
Bind Preferentially to Angiostatin protein
over Plasminogen

| Peptide | Amino Acid Sequence | SEQ ID NO: |
|---|---|---|
| PAC-77 | C W S L E H S K C G G G S | SEQ ID NO:38 |
| PAC-78 | C V H S I E R E C G G G S | SEQ ID NO:39 |
| PAC-82 | C Y T L P P K L C G G G S | SEQ ID NO:40 |
| PAC-88 | C W S Y E W S K C G G G S | SEQ ID NO:41 |
| PAC-91 | C W S L E W Q K C G G G S | SEQ ID NO:42 |

### EXAMPLE 3

Comparison of Angiostatin Binding Peptides using Phage Display Technology and ELISA Assays

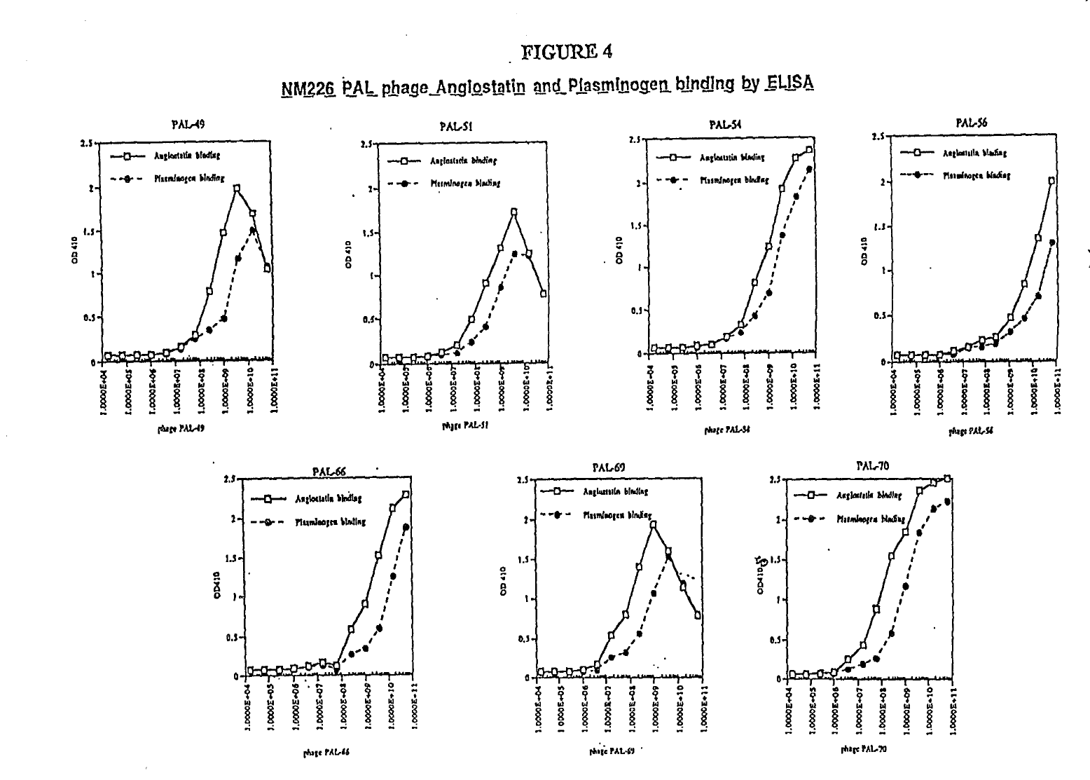

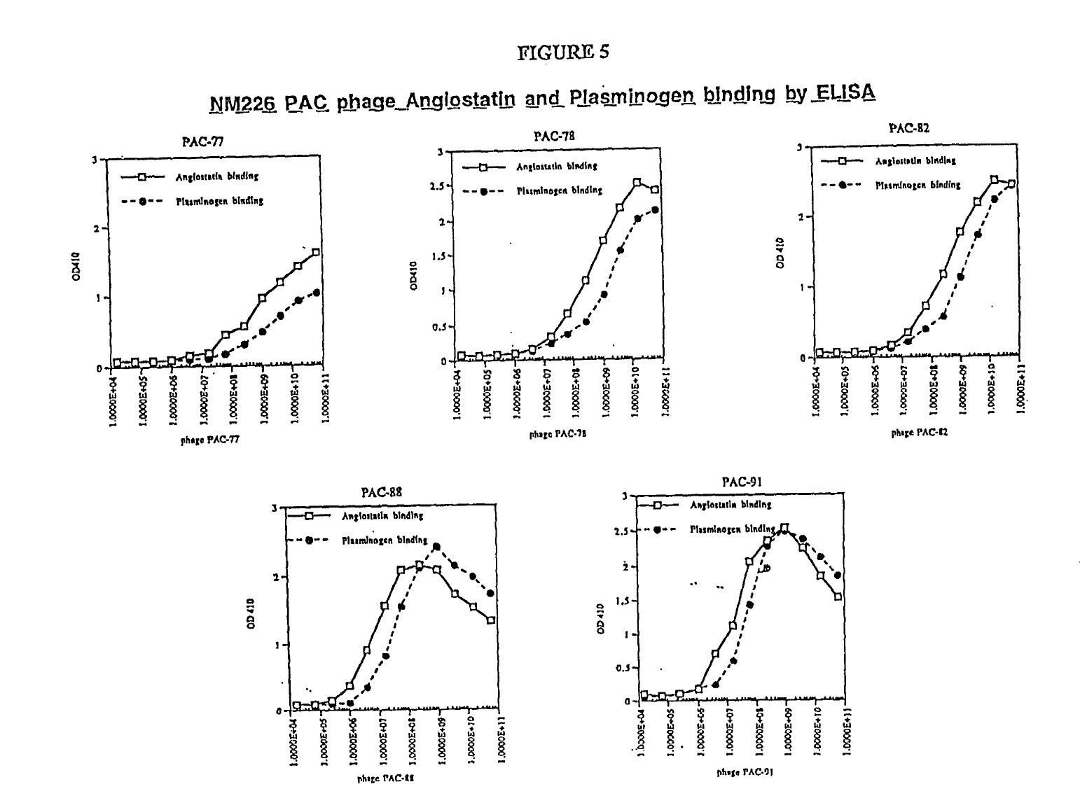

A modified ELISA assay was used to determine the specificity of binding of the phage to the target protein as recommended by New England BioLabs. Plates coated with the target proteins (Angiostatin protein, Endostatin, or plasminogen) were incubated with four-fold serial dilutions of the phage from approximately 6.25×10¹⁰ to 2×10⁵ phage particles in 96-well plates. The plates were washed six times with TBS containing 0.5% Tween 20 followed by incubation with 1:5000 diluted HRP-conjugated anti-M13 antibody (Pharmacia # 27-9411-01). The plates were washed six times with TBS containing 0.5% Tween 20 followed by incubation with ABTS (2,2³-AZINO-bis(3-ETHYLBENZ-THIAZOLINE-6 SULFONIC ACID)) Peroxidase substrate solution at room temperature for 10-60 minutes. The plates were read at 410 nm using a Molecular Devices Spectra MAX 250 microplate reader and SOFTmax® Pro software. As shown in FIGS. 4 and 5, the peptides selected using phage display technology, preferentially bound to target proteins Angiostatin protein over plasminogen.

### EXAMPLE 4

Identification of Proteins Bearing Sequence Homology to Angiostatin Protein Binding Peptides or Endostatin Protein Binding Peptides

A protein database was searched for proteins that share sequence homology with the peptides identified previously as Angiostatin protein and Endostatin protein binding peptides (Peptide numbers A1-A11, E12-E22, A25-A36, E37-E48, PAL-49, 51, 54, 56, 66, 69, 70, and PAC-77, 78, 82, 88 and 91). Because of the short length of the peptides, a significant number of proteins that shared homology with the identified peptides were found. A selection of the peptides with the highest homology or those that appear to be biologically interesting are provided in Tables 7 and 8. The selection of proteins with the highest homology with the PAL and PAC peptides or those that appear to be biologically interesting are provided in Tables 9 and 10. Homology searches were conducted using the FASTA sequence similarity search. The peptide sequences were queried against the SwissProt data base using the default setting (ktup 2, and BLOSUM50 for the scoring matrix). The searches were conduced using the world wide web at site fasta.genome.adjp/. as described by W. R. Pearson & D. J. Lipman PNAS 85:2444-2448 (1988).

TABLE 7
Search Results for Homologies with the Linear Angiostatin
Binding Peptides and Endostatin Binding Peptides

| Protein Containing | | |
|---|---|---|
| Homologous Sequence | Source | Level of Homology |
| Human possible global | Mammalian | 7 of 7 amino acids with A9 |
| transcription activator | (Human) | |
| phosphopentomutase | Bacteria | 6 of 7 amino acids plus 1 |
| | | conserved with A3 |
| ribonuclease RH | Fungi | 6 of 7 amino acids plus 1 |
| precursor | | conserved with A35 |
| soybean early nodulin | Soybean | 6 of 7 amino acids plus 1 |
| | | conserved with A25 |
| JNK activating | Mammalian | 6 of 7 amino acids with A3 |
| kinase 1 | (Human) | |
| IL-12 beta chain | Mammalian | 6 of 7 amino acids with A34 |
| | (Mouse) | |
| glutathione reductase | Spinach | 5 of 7 amino acids plus 2 |
| | (Chloroplast) | conserved with A3 |
| Soy bean trypsin | Soybean | 5 of 7 plus 1 conserved |
| inhibitor (kunitz) | | amino acid with A1 |
| fibroblast growth | Human | 5 of 7 plus 2 conserved |
| factor-6 | Mouse | amino acid with E18 |
| chemotaxis protein | Bacteria | 5 of 7 plus 1 conserved |
| | | amino acid with E12 |
| Annexin XI | Human | 5 of 7 plus 1 conserved |
| | Rabbit | amino acid with E22 |
| | Mouse | |
| WEE 1 | Yeast | 5 of 7 plus 1 conserved |
| | | amino acid with A32 |
| RAS suppressor | Mammalian | 5 of 7 with A32 |
| protein 1 | (Human) | |
| ATP synthase gamma | Bacteria | 5 of 7 with A32 |
| chain | | |
| Thioredoxin | Bacteria | 5 of 7 with A1 |

TABLE 8
Search Results for Homologies with the Constrained Angiostatin
Binding Peptides and Endostatin Binding Peptides

| Protein Containing | | |
|---|---|---|
| Homologous Sequence | Source | Level of Homology |
| Collagenese* | Bacteria | 9 of 13 with A32 |
| Glycoprotein B-1 | Virus | 8 of 11 with E12 |
| precursor | | |
| Dehydroquinate | Bacteria | 8 of 11 with E16 |
| dehydratase | | |
| Complement | Mammalian | 8 of 13 with plus 2 conserved |
| component C8 beta | (Rat) | with A25 |
| chain* | | |
| Ornithine | Mammalian | 7 of 11 plus 2 conserved with |
| decarboxylase | (Hamster) | E13 |
| antizyme | | |
| adenylate cyclase | Bacteria | 7 of 11 plus 2 conserved with |
| | | E20 |
| ATP synthase alpha | Bacteria | 6 of 11 plus 4 conserved with |
| chain | | A1 |
| ATP synthase alpha | Bacteria | 6 of 11 plus 1 conserved with |
| chain | | A10 |

*The constrained peptides were 13 amino acids because of the presence of the 2 cysteines i.e. C-XXXXXXX-CGGGS.

An additional selection of peptides having high homology and appearing biologically interesting, are provided in Tables 9 and 10.

TABLE 9
Additional Search Results for Homologies with the Linear
Angiostatin Binding Peptides and Endostatin Binding Peptides

| Protein Containing | | |
|---|---|---|
| Homologous Sequence | Source | Level of Homology |
| FLIY PROTEIN | E. coli | 6 out of 7 plus 1 conserved |
| PRECURSOR | bacteria | with PAL-66 |
| HEMAGGLUTININ- | Para | 6 out of 7 plus 1 conserved |
| NEURAMINIDASE | influenza | with PAL-66 |
| | (Flu Virus) | |
| HISTONE H1 | Drosphila | 6 out of 7 PAL-56 |
| | (fruit fly) | |
| ADP, ATP CARRIER | Wheat (plant) | 6 out of 7 plus with PAL-66 |
| PROTEIN 2 | | |
| PRECURCOR | | |
| RIBONUCLEASE 1 | Thale-cress | 5 out of 7 plus 2 conserved |
| PRECURSOR | (plant) | with PAL-51 |
| CALRETININ | Mouse | 5 out of 7 plus 2 conserved |
| | (mammalian) | with PAL-53 |
| HYPOTHETICAL | Yeast | 5 out of 7 plus 2 conserved |
| 64.1 KD ZINC | (eukaryotic) | with PAL-70 |
| FINGER PROTEIN | | |
| T-CELL SURFACE | Rat and | 5 out of 7 plus 2 conserved |
| ANTIGEN CD2 | mouse | with PAL-70 |
| PRECURSOR | (mammalian) | |
| ATP SYNTHASE C | Bacillus | 5 out of 7 plus 1 conserved |
| CHAIN | alcalophilus | with PAL-66 |
| | (bacteria) | |
| CELL DIVISION | E. coli | 5 out of 7 plus 1 conserved |
| INHIBITOR MINC. | (bacteria) | with PAL-56 |
| CELL DIVISION | Yeast | 5 out of 7 plus 1 conserved |
| CONTROL PROTEIN | (eukaryotic) | with PAC-78 |
| 45 | | |
| FETAL BRAIN | Human | 5 out of 7 plus 1 conserved |
| PROTEIN | (mammalian) | with PAL-53 |
| HOLLIDAY | Pseudomonas | 5 out of 7 plus 1 conserved |
| JUNCTION DNA | aeruginosa | with PAL-53 |
| HELICASE RUVA | (bacteria) | |

TABLE 10
Additional Search Results for Homologies with the Constrained
Angiostatin Binding Peptides and Endostatin Binding Peptides

| Protein Containing | | |
|---|---|---|
| Homologous Sequence | Source | Level of Homology |
| CELL WALL | Trichoderma | 8 of 13 plus 2 conserved with |
| PROTEIN QID3 | harzianum | PAC-82* |
| PRECURSOR | (eukaryotic) | |
| HYDROPHOBIN | Aspergillus | 8 of 11 plus 1 conserved with |
| PRESURSOR | fumigatus | PAL-66 |
| (RODLET PROTEIN) | (Eukaryotic) | |
| CARBOXYPEPTIDAS | Yeast | 7 of 13 plus 4 conserved with |
| E Y PRECURSOR | (eukaryotic) | PAC-78* |
| NODULIN 24 | Soy Bean | 7 of 11 plus 2 conserved with |
| PRECURSOR | (plant) | PAL-51 |
| HYPOTHETICAL | E. coli | 7 of 11 plus 1 conserved with |
| ABC TRANSPORTER | (bacteria) | PAL-66 |
| PERMEASE PRO | | |

*The constrained peptides were 13 amino acids because of the presence of 2 cysteines i.e. C-XXXXXXX-CGGGS.

### EXAMPLE 5

### Identification of Angiostatin and Endostatin Binding Proteins

Based upon the rationale that the peptides that bound to Angiostatin protein or Endostatin protein represent the binding domains of larger proteins that bind Angiostatin and/or Endostatin proteins, it was theorized that Angiostatin protein and/or Endostatin protein binding peptides and proteins would share common motifs that are recognized by corresponding anti-peptide antibodies. Consequently, a HUVEC expression library was screened with antibodies against the A10 and E37 peptides (A10 peptide binds Angiostatin protein while E37 peptide binds Endostatin protein, see Examples 1 and 2). In doing so, multiple clones that code for human tropomyosin were isolated. Though not wishing to be bound by the following theory, it is thought that it is the anti-E37 antibodies recognize tropomyosin with greater affinity than the anti-A10.

The multiple clones that code for human tropomyosin were identified as follows. The 5 prime and 3 prime ends of the clones recognized by the A10 and E37 antibodies were determined by routine DNA sequencing techniques. The obtained sequences were then ‘BLASTED’ against the GeneBank database (of DNA sequences) and a high degree of sequence similarity was observed between both the 5 prime and 3 prime end sequences of antibody-recognized clone and human tropomyosin mRNA. The BLAST searches were conducted using the website at http://www.blast.genome.adjpl using the default setting (scoring matrix BLOSUM62). Results of the BLAST searches are provided below.

5 Prime End Blast Results

High Probability

Sequences producing High-scoring Segment Pairs: Score P(N) N

gb:HUMTRO Human tropomyosin mRNA, complete cds. 342 2.1e-20 1

gb:S78854 alpha-tropomyosin [rabbits, New Zealand whi . . . 309 1.1e-17 1

gb:SSCATROP S.scrofa mRNA for cardiac alpha tropomyosin. 302 4.3e-17 1

gb:HUMTROPA2 Human skeletal muscle alpha-tropomyosin (hT . . . 297 1.1e-16 1

gb:RATTMBR1A Rat brain alpha-tropomyosin (TMBr-1) mRNA, . . . 293 2.4e-16 1

gb:MMPTMA M.musculus mRNA for skeletal muscle alpha t . . . 293 2.4e-16 1

gb:MUSTRO2IS Mouse tropomyosin isoform 2 mRNA, complete . . . 293 2.5e-16 1

gb:RATTRO2A Rat alpha-tropomyosin 2 mRNA, complete cds. 293 2.5e-16 1

gb:RATTRO3A Rat alpha-tropomyosin 3 mRNA, complete cds. 293 2.5e-16 1

gb:RATTMA1 Rat alpha-tropomyosin gene, exons 1-3. 279 3.9e-15 1

gb:AR008277 Sequence 3 from U.S. Pat. No. 5,753,446. 242 8.7e-15 2

gb:AR008281 Sequence 11 from U.S. Pat. No. 5,753,446. 242 8.7e-15 2

gb:RATTROPA Rat smooth muscle alpha-tropomyosin mRNA, c . . . 268 1.3e-13 1

gb:RNTROASM Rat mRNA for smooth muscle alpha-tropomyosin. 268 1.3e-13 1

gb:A62300 Sequence 3 from Patent WO9712982. 228 1.2e-12 2

gb:HUMTRPMYO Human tropomyosin-1 (TM-beta) mRNA, complet . . . 246 3.1e-11 1

gb:HUMTM1E H.sapiens epithelial tropomyosin (TM1) mRNA . . . 246 3.1e-11 1

gb:RATTRO01 Rat skeletal muscle beta-tropomyosin and fi . . . 237 2.2e-10 1

gb:A62298 Sequence 1 from Patent WO9712982. 234 4.7e-10 1

gb:AR018138 Sequence 1 from U.S. Pat. No. 5,780,609. 234 4.8e-10 1

gb:AR008278 Sequence 5 from U.S. Pat. No. 5,753,446. 230 1.1e-09 1

gb:AMTTROPOX Ambystoma mexicanum tropomyosin mRNA, compl. 229 1.3e-09 1

gb:AB005878 Nicotiana tabacum mRNA for BYJ15, partial cds. 228 1.4e-09 1

gb:AB005879 Nicotiana tabacum mRNA for BYJ6, partial cds. 228 1.4e-09 1

gb:DMELAST D.melanogaster mRNA for elastin-like protein. 228 1.4e-09 1

gb:TEZ86120 T.evansi mRNA, clone Q16R1. 228 1.6e-09 1

gb:MMBTROP M.musculus mRNA for beta-tropomyosin. 228 1.6e-09 1

gb:MUSBETATRO Mouse beta-tropomyosin 2 mRNA, complete cds. 228 1.6e-09 1

gb:MMTPMYOB Mus musculus gene for beta-tropomyosin. 228 1.6e-09 1

gb:AB002449 Homo sapiens mRNA from chromosome 5q21-22, . . . 228 1.6e-09 1

gb:OSCHINDPR O.sativa mRNA for chilling-inducible protein. 228 1.7e-09 1

gb:I14842 Sequence 10 from U.S. Pat. No. 5,455,167. 228 1.7e-09 1

gb:I65496 Sequence 6 from U.S. Pat. No. 5,667,997. 228 1.7e-09 1

gb:ATCYC2B A.thaliana (Columbia) cyc2b mRAN for cyclin . . . 228 1.7e-09 1

gb:I79511 Sequence 2 from U.S. Pat. No. 5,707,809. 228 1.7e-09 1

gb:AF060519 Cuphea hookeriana 3-ketoacyl-ACP synthase ( . . . 228 1.7e-09 1

gb:MUSTROB Mouse skeletal muscle beta tropomyosin mRNA . . . 228 1.7e-09 1

gb:AF058696 Homo sapiens cell cycle regulatory protein . . . 228 1.8e-09 1

gb:MSASET2MR M.sativa mRNA for ASET2. 220 8.9e-09 1

gb:AB005877 Nicotiana tabacum mRNA for BYJ14, partial cds. 219 9.0e-09 1

gb:S71728 truncated protein {frame 1, multiple clonin . . . 198 9.4e-09 1

gb:I50126 Sequence 19 from U.S. Pat. No. 5,641,876. 198 1.1e-08 1

gb:I50132 Sequence 25 from U.S. Pat. No. 5,641,876. 198 1.11e-08 1

gb:I50128 Sequence 21 from U.S. Pat. No. 5,641,876. 198 1.1e-08 1

gb:I50133 Sequence 26 from U.S. Pat. No. 5,641,876. 198 1.2e-08 1

gb:PSP54MRNA Pisum sativum mRNA for P54 protein. 218 1.4e-08 1

gb:CHKTRPMYB Chicken skeletal muscle beta-tropomyosin mR . . . 216 2.1e-08 1

gb:CHKTROBRT1 Chicken beta-tropomyosin 1 (BRT-1) mRNA, co . . . 216 2.2e-08 1

gb:I82448 Sequence 60 from U.S. Pat. No. 5,712,143. 213 2.2e-08 1

gb:CHKTROSS01 Chicken tropomyosin beta subunit gene, exon . . . 216 2.2e-08 1

gb:AR013938 Sequence 1 from U.S. Pat. No. 5,773,225. 193 3.4e-08 1

gb:AR016808 Sequence 33 from U.S. Pat. No. 5,777,200. 213 3.7e-08 1

gb:AR020834 Sequence 33 from U.S. Pat. No. 5,789,214. 213 3.7e-08 1

gb:I38463 Sequence 33 from U.S. Pat. No. 5,614,395. 213 3.7e-08 1

gb:I56938 Sequence 33 from U.S. Pat. No. 5,650,505. 213 3.7e-08 1

gb:I59804 Sequence 33 from U.S. Pat. No. 5,654,414. 213 3.7e-08 1

gb:I75131 Sequence 33 from U.S. Pat. No. 5,689,044. 213 3.7e-08 1

gb:NTZ82982 N.tabacum mRNA for caffeoyl-CoA 0-methyltra . . . 213 3.9e-08 1

gb:MZEAGAMOU Zea maize AGAMOUS homologue mRNA, complete 213 3.9e-08 1

gb:AF030383 Cucumis melo var. markuwa Markino ADP-gluco . . . 213 4.0e-08 1

gb:CLCYCBMR C.longicaudatus mRNA for cyclin B. 213 4.0e-08 1

gb:CPU69698 Cryptosporidium parvum heat shock protein 7 . . . 211 6.2e-08 1

gb:S81027 Msr-110=EN protein binding gene/engrailed n . . . 208 1.1e-07 1

gb:HUMTROPA Human fibroblast muscle-type tropomyosin mR . . . 207 1.3e-07 1

gb:CHKATRO Chicken fast-twitch alpha-tropomyosin mRNA, . . . 207 1.3e-07 1

gb:HSTPMYOB Human mRNA for skeletal beta-tropomyosin. 207 1.3e-07 1

gb:QULTROAB Japanese quail alpha-tropomyosin mRNA, comp . . . 207 1.3e-07 1

gb:I94990 Sequence 9 from U.S. Pat. No. 5,731,411. 188 1.3e-07 1

gb:CHKAFTROP1 Chicken alpha-tropomyosin gene, exons 1a, 2 . . . 207 1.4e-07 1

gb:GGTME1A2A G.gallus gene for tropomyosin (alpha isofor . . . 207 1.4e-07 1

gb:CCTPMY01 Quail gene for alpha-tropomyosin, exons 1-3 . . . 207 1.4e-07 1

gb:QULTROAA Japanese quail alpha-tropomyosin mRNA, comp . . . 206 1.7e-07 1

gb:MAU29167 Mesocricetus auratus tropomyosin-1 mRNA, co . . . 205 2.0e-07 1

gb:S71730 influenza virus hemagglutinin 5′ epitope ta . . . 198 2.0e-07 1

gb:DMIPOU D.melanogaster I-POU mRNA for a POU-domain . . . 205 2.1e-07 1

gb:SYNPLKRA Cloning vector pUC128 DNA, polylinker region. 198 2.4e-07 1

gb:SYNPLKRB Cloning vector pUC129 DNA, polylinker region. 198 2.6e-07 1

gb:S71742 influenza virus hemagglutinin 5′ epitope ta . . . 198 2.8e-07 1

gb:A59058 Sequence 1 from Patent WO9703200. 203 3.1e-07 1

gb:GGINTB3 G.gallus mRNA for integrin beta3. 203 3.2e-07 1

gb:STRSTRH Streptococcus pneumoniae beta-N-acetylhexos . . . 203 3.3e-07 1

gb:S71745 influenza virus hemagglutinin 5′ epitope ta . . . 198 3.5e-07 1

gb:RANATROA R.temporaria skeletal muscle alpha-tropomyo . . . 201 4.5e-07 1

gb:AF067142 Cloning vector pSFI polylinker, complete po . . . 198 4.6e-07 1

gb:AR016514 Sequence 24 from U.S. Pat. No. 5,776,746. 198 6.0e-07 1

gb:CVU61229 Cloning vector pKRX, complete sequence. 199 7.3e-07 1

gb:AR008443 Sequence 70 from U.S. Pat. No. 5,753,488. 198 7.8e-07 1

gb:I87435 Sequence 70 from U.S. Pat. No. 5,703,221. 198 7.8e-07 1

gb:PFL17187 Platichthys flesus Ki-ras gene (exons 1, 2, . . . 198 7.9e-07 1

gb:DMFRIZZ3 D. melanogaster frizzled gene exons 3 and 4. 198 8.4e-07 1

gb:SMOFMTRMYA Salmo salar fast myotomal muscle tropomyosi . . . 198 8.4e-07 1

gb:CHKTROPB Chicken tropomyosin (clone CTm7) mRNA, comp . . . 198 8.5e-07 1

gb:A45456 Sequence 1 from Patent EP0607925. 198 8.5e-07 1

gb:I7500 Sequence 1 from U.S. Pat. No. 5,489,743. 198 8.5e-07 1

gb:D88747 Arabidopsis thaliana mRNA for AR401, comple . . . 198 8.6e-07 1

gb:NTY14032 Nicotiana tabacum mRNA for ferredoxin-NADP . . . 198 8.6e-07 1

gb:CHKTROPA Chicken tropomyosin (clone CTm4) mRNA, comp . . . 198 8.6e-07 1

gb:MPCAM M.pyrifera mRNA for calmodulin. 198 8.7e-07 1

gb:PFL17188 Platichthys flesus Ha-ras gene (exons 1 to 4). 198 8.7e-07 1

gb:ORZPC Oryzias latipes mRNA for photolyase, comple . . . 198 8.8e-07 1

3 Prime End Blast Results

Sequences producing High-scoring Segment Pairs: Score P(N) N

gb:HSAJ147 Homo sapiens mRNA for alpha-tropomyosin (3′ . . . 700 1.9e-110 4

gb:HSALPTROP Homo sapiens mRNA for alpha-tropomyosin. 1055 1.4e-96 2

gb:HSTROISOA H.sapiens tropomyosin isoform mRNA, complet . . . 1012 2.9e-76 1

gb:MUSTRO2IS Mouse tropomyosin isoform 2 mRNA, complete . . . 226 4.3e-33 4

gb:RATTMA6 Rat alpha-tropomyosin gene, exon 12. 267 1.9e-28 3

gb:RATTRO5A Rat alpha-tropomyosin 5a mRNA, complete cds. 267 7.9e-24 3

gb:RATTRO5B Rat alpha-tropomyosin 5b mRNA, complete cds. 267 7.9e-24 3

gb:RATTRO2A Rat alpha-tropomyosin 2 mRNA, complete cds. 267 8.7e-24 3

gb:RATTRO3A Rat alpha-tropomyosin 3 mRNA, complete cds. 267 8.7e-24 3

gb:RATTMBR3A Rat brain alpha-tropomyosin (TMBr-3) mRNA, . . . . 267 2.2e-23 3

gb:RATTROPA Rat smooth muscle alpha-tropomyosin mRNA, c . . . 206 5.9e-18 3

gb:RNTROASM Rat mRNA for smooth muscle alpha-tropomyosin. 206 5.9e-18 3

gb:CHKAFTROP6 Chicken alpha-tropomyosin gene, exon 9c, 9d . . . 273 2.9e-12 1

gb:GGTME9C9D G.gallus gene for tropomyosin (alpha isofor . . . 273 2.9e-12 1

gb:HSTROPM Human mRNA (exon 6-9 part.) for smooth musc . . . 224 3.6e-08 1

gb:HUMTROPA4 Human skeletal muscle alpha-tropomyosin (hT . . . 224 3.6e-08 1

gb:HUMTRO Human tropomyosin mRNA, complete cds. 224 3.7e-08 1

gb:CCTPMY13 Quail gene for alpha-tropomyosin, exon 13. 146 0.13 1

gb:DMU66884 Drosophila melanogaster cubitus interruptus . . . 133 0.83 1

gb:CEZK666 Caenorhabditis elegans cosmid ZK666, comple . . . 132 0.88 1

gb:AC004002 Human BAC clone RG356F09 from 7p21, complet . . . 132 0.88 1

As a result of the findings of the above experiments, nine clones from a HUVEC lambda Uni-ZAP XR cDNA library were purified based on the observation that they express a protein that is recognized by polyclonal antibodies raised against the A10 and E37 peptide (see Examples 1 and 2). Sequencing data showed that 6 of the 9 code for tropomyosin protein. The sequencing reaction for one clone failed but restriction analysis showed that it also codes for tropomyosin. Thus, 7 of the 9 clones code for tropomyosin.

Additionally, analysis of the DNA sequence of one clone showed it to code for human glyceraldehyde 3-phosphate dehydrogenase (GAPDH) at the 5′ end and for an SH3 domain binding glutamic acid-rich-like protein at the 3 prime end. The GenBank accession number for these sequences is HUMG3 PDC and AF042081 respectively. Another clone shares homology with (cr36c10.x1) Jia bone marrow stroma Homo sapiens cDNA clone. The GenBank accession number for this sequence is AI755161.

### EXAMPLE 6

E37 Peptide Represents a Mimotope of the Endostatin Protein-binding Domain of hTM3

A comparison of the primary sequence of hTM3 with that of the E37 peptide failed to reveal homology, thus suggesting that the E37 peptide sequence represents a mimotope of the Endostatin-binding region of tropomyosin. To confirm that the E37 peptide shared an epitope with tropomyosin, immunoblots were run using E. coli lysate prepared from LacZ-tropomyosin fusion protein expressing cells. Briefly, plasmid pλ21-1 was excised as a pBluescript phagemid from a recombinant Uni-ZAP XR phage isolated from the HUVEC cDNA library based on its recognition by the E37 anti-sera. DNA sequencing of the cDNA insert of pλ21-1 showed that it contained the complete human tropomyosin coding sequence fused in frame downstream of the Lac Z gene. E. coli DH5α cells were transformed with pλ21-1 and pBluescript. Crude bacterial lyastes were prepared from IPTG induced cultures of pλ21-1 (λ21-1 lysate) and pBluescript (control lysate) transformed DH5a cells following the procedure described in Ausubel et al., Short protocols in molecular biology: a compendium of methods from Current protocols in molecular biology (Greene Pub. Associates; Wiley, New York, N.Y., ed. 3rd, 1995).

The E. coli lysates were probed with either a monoclonal antibody raised against chicken gizzard tropomyosin (TM311) or the E37 anti-peptide sera (data not shown). The anti-E37 sera and TM311 anti-tropomyosin antibody recognized common bands of approximately 49 and 43 kDa in the recombinant −hTM3 expressing λ21-1 lysate, neither of which were detected in the control lysate. Furthermore, when HUVEC lysate was probed with anti-E37 sera or TM311 anti-tropomyosin antibody, a common band of approximately 38 kDa was identified (data not shown). These findings demonstrate that the E37 peptide and hTM3 share a common epitope. Surprisingly, although the TM311 anti-tropomyosin antibody recognized tropomyosin purified from human cardiac tissue, rabbit muscle and chicken muscle, the anti-E37 sera showed no specificity for these isoforms of tropomyosin (data not shown). The amino acid sequences of rabbit muscle and human cardiac tropomyosins share 86% identity with hTM3 diverging in only two regions, between amino acids 189 and 212, and at the carboxy terminus from amino acids 258 to 284. This observation suggests that the putative Endostatin protein-binding domain of hTM3 resides within one of these divergent regions located in the carboxy third of hTM3. These results suggest that Endostatin protein does not interact with all isoforms of tropomyosin and that Endostatin protein may display subtype specificity in its tropomyosin-binding properties to inhibit the functions of some but not all isoforms of tropomyosin.

### EXAMPLE 7

In vitro Interaction of Tropomyosin and Endostatin Protein

The following experiment was conducted in order to assess the interaction of tropomyosin and Endostatin protein.

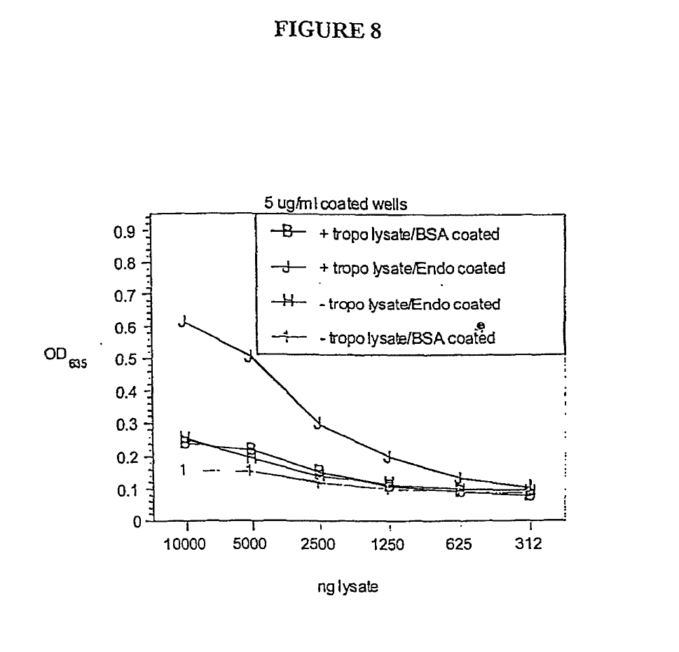

In this experiment the wells of a 96 well plate were either coated with 5 μg/ml of Endostatin protein or BSA protein. The wells were incubated in the presence of serial dilutions of E. coli lysate that either contained or did not contain tropomyosin protein. The wells were washed and incubated with anti-tropomyosin antibodies followed by anti-mouse IgG AP conjugated antibodies and developed using KPL Blue Phos microwell phosphatase solution. The OD₆₃₅ was measured after a one hour incubation at room temperature and the results plotted in FIG. 8.

As shown in FIG. 8, when E. coli lysate containing tropomyosin was incubated on an Endostatin protein coated well (sample 2), considerably more tropomyosin bound than when the well was BSA coated or when there was no tropomyosin contained in the E. coli lysate. The results of this experiment therefore demonstrate positive binding interaction between tropomyosin and Endostatin protein.

### EXAMPLE 8

Role of E37 Binding Peptide on Anti-tumor Activity of Endostatin Protein

The following experiment was conducted to determine the interaction of E37 peptide and Endostatin protein. Briefly, the B16BL6 experimental metastasis assay, as known to those of skill in the art and further described in International Patent Application number PCT/US99/11418, was performed as follows. 5×10⁴ B16-BL6 cells in 200 μl of PBS were injected into C57BL/6J mice via the tail vein. Three days post injection treatment was started. The injection received by eight groups of mice were: 1) citrate phosphate/PBS buffer, 2) ³⁰ μg Endostatin protein (in 100 μl PBS), 3) 30 μg Endostatin protein (in 200 μl PBS), 4) 30 μg Endostatin protein plus 50 times molar excess of control peptide (in 200 μl PBS), 5) 30 μg Endostatin protein plus 250 times molar excess of control peptide (in 200 μl PBS), 6) 30 μg Endostatin protein plus 10 times molar excess of E37 peptide (in 200 μl PBS), 7) 30 μg Endostatin protein plus 50 times molar excess of E37 peptide (in 200 μl PBS), and 8) 30 μg Endostatin protein plus 250 times molar excess of E37 peptide (in 200 μl PBS). Doses were administered daily for eleven days posterior to the thoracic cavity subcutaneously. All mice were sacrificed, the lungs removed and the number of surface metastases in each animal were counted under a stereo microscope.

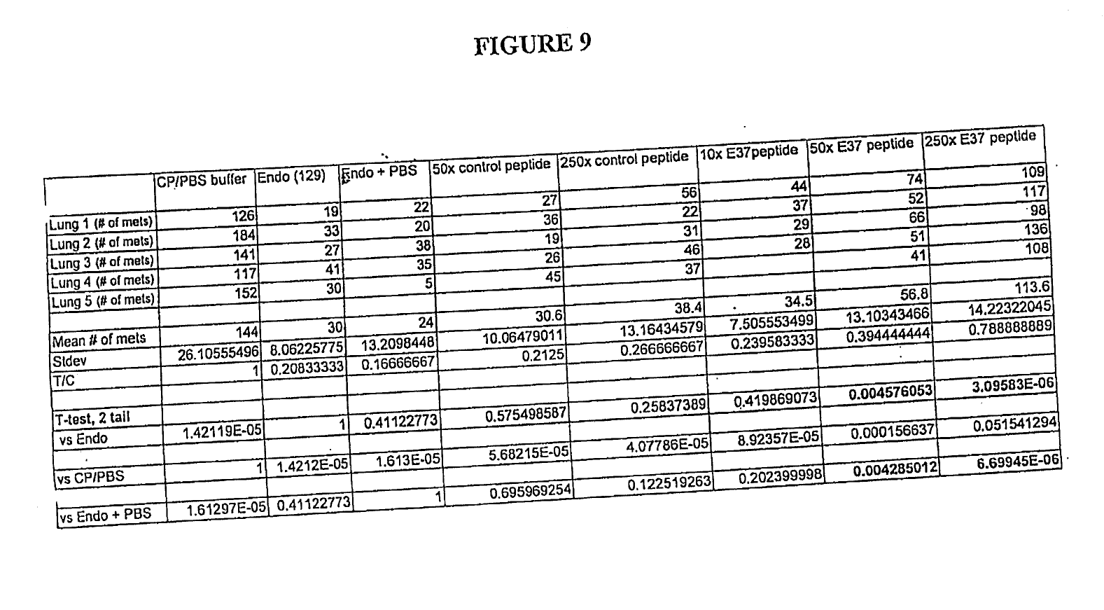

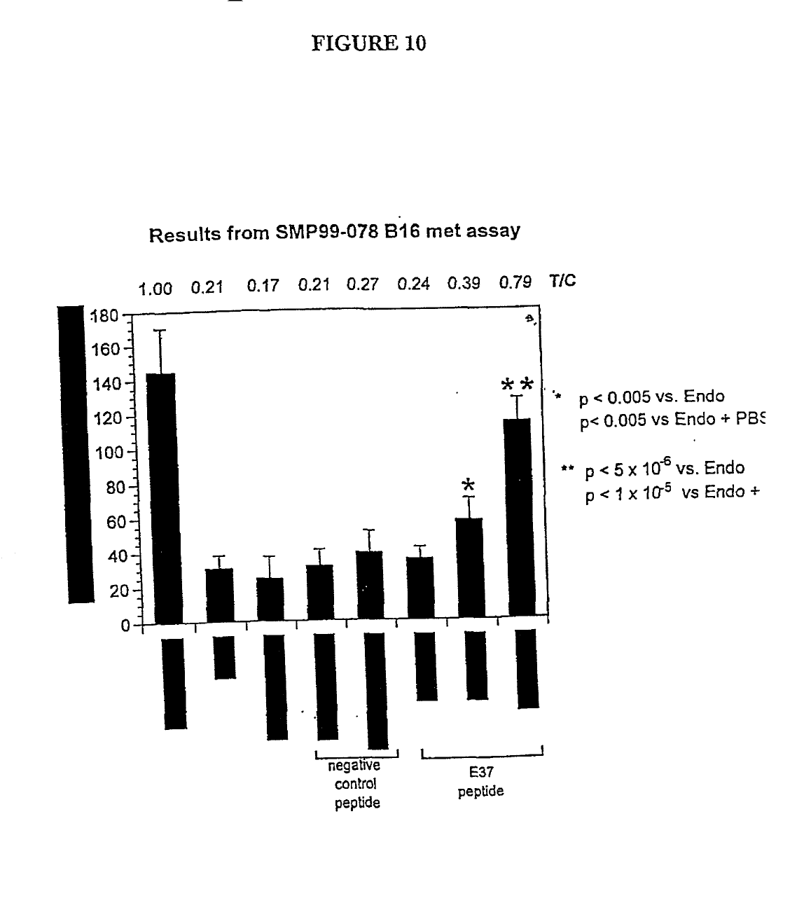

A summary of the results of this experiment are briefly described below. The detailed results of this experiment are provided in FIG. 9 wherein the number of metastases on each lung is listed, as well as the mean, standard deviation, T/C and the results of a 2-tailed T-test. The results are graphically described in FIG. 10. Briefly, Endostatin protein alone inhibited the formation of lung metastasis by approximately 79%. Endostatin protein plus a 50 fold molar excess of the negative control peptide inhibited the formation of lung metastasis by approximately 79%. Endostatin protein plus a 250 fold molar excess of the negative control peptide inhibited the formation of lung metastasis by approximately 73%. Endostatin protein plus a 10 fold molar excess of the E37 peptide inhibited the formation of lung metastasis by approximately 76%. Endostatin protein plus a 50 fold molar excess of the E37 peptide inhibited the formation of lung metastasis by approximately 61%. Endostatin protein plus a 250 fold molar excess of the E37 peptide inhibited the formation of lung metastasis by approximately 21%.

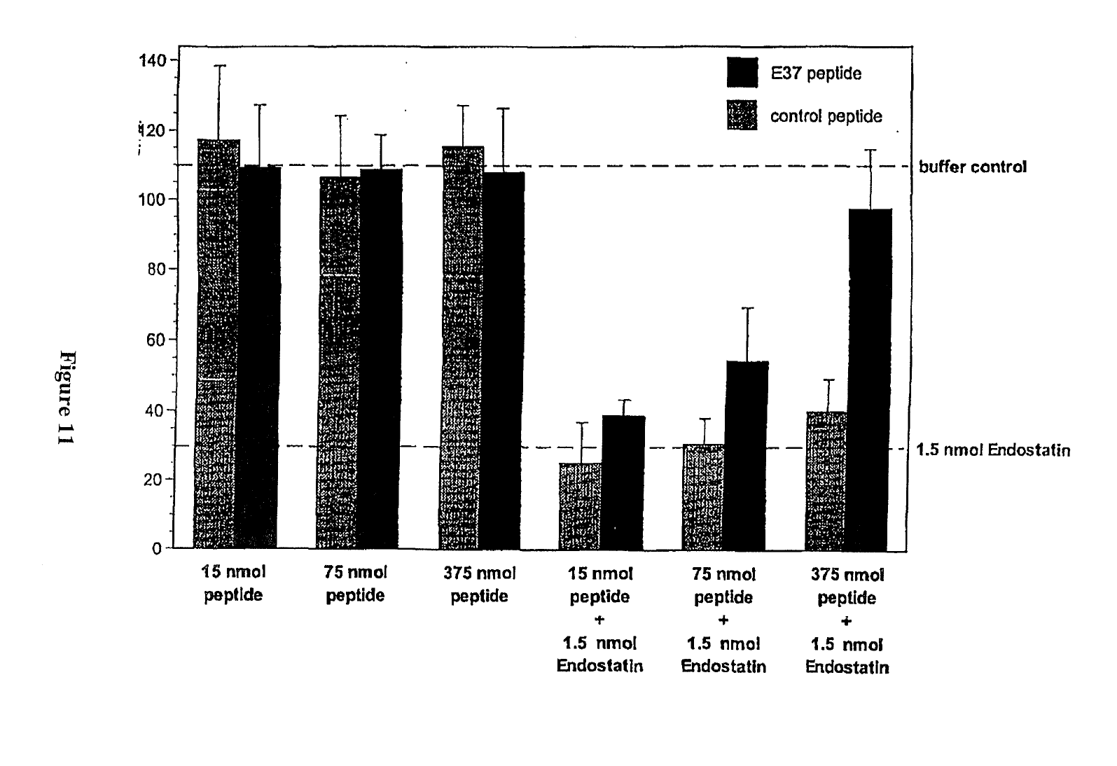

In another experiment, the role of the E37 mimotope in the anti-tumor activity of rhEndostatin protein was assessed as follows. B16-BL6 mice harboring lung metastases were treated with a daily administration of 1.5 nmol of rhEndostatin protein (1.5 mg/kg/day). It was determined that the 1.5 mg/kg/day of rhEndostatin inhibited the growth of experimental B16-BL6 lung metastases by greater than 70% as compared to buffer treated control mice (FIG. 11). Subsequently B16-BL6 mice were treated with up to 375 nmol/day of E37 peptide in the absence of rhEndostatin protein and it was determined that the number of metastatic lesions on the lungs of the mice was not statistically different from that of the control group. This data demonstrated that the E37 peptide alone has neither tumor promoting or inhibitory activity in this assay.

However, co-administration of rhEndostatin protein with E37 peptide dramatically inhibited the anti-tumor activity of the rhEndostatin protein. Co-injection of 1.5 nmol of rhEndostatin protein with 15, 75 and 375 nmol of the E37 peptide resulted in a dose dependent decrease in the anti-tumor activity of the Endostatin protein of 11, 30.5 and 84.5 percent respectively. The number of pulmonary metastases in mice co-treated with rhEndostatin protein and either 75 or 375 nmol of E37 peptide (50 and 250 fold molar excess respectively) were significantly lower than that on the lungs of the rhEndostatin protein alone treated mice (p<0.02). The incidence of metastatic lesions on the lungs of mice co-injected with 1.5 nmol rhEndostatin protein and up to 375 nmol of a cyclic disulfide bonded control peptide (CSNSDKPKCGGGS) (SEQ ID NO:118) or in mice treated with 1.5 nmol of the rhEndostatin protein alone was statically indistinguishable (p>0.11). Thus, in the experimental B16-BL6 lung metastases model, the anti-tumor activity of the rhEndostatin protein is specifically blocked in a dose dependent manner by co-injection of E37 peptide. Although not wanting to be bound by this theory, it is believed that the E37 peptide competes with tropomyosin for the binding of rhEndostatin protein, resulting in disruption of the tropomyosin-rhendostatin interaction, an interaction that is crucial for the anti-tumor activity of rhEndostatin protein.

### EXAMPLE 9

Effect of Endostatin Protein on Pericytes and Fibroblasts

Endostatin protein results in an inhibition of proliferation of PDGF-BB or 20% fetal calf serum stimulated primary cultures of pericytes and to a lesser extent fibroblasts. More specifically, Endostatin protein results in a consistent reduction in thymidine incorporation into primary cultures of pericytes and to a lesser degree fibroblasts, that are stimulated to proliferate in response to PDGF-BB or 20% fetal calf serum. The results have been confirmed using a MTT proliferation kit.

Furthermore, Endostatin protein does not appear to affect the differentiation of pericytes to fibroblasts, although inhibition of proliferation varies depending to the degree of differentiation along the pericyte/fibroblast differentiation pathway. Endostatin protein tends to have a greater effect on cells in the earlier stages of differentiation. While such cells constitute a small fraction of the total number, inhibiting their proliferation may have significant effects on the final total cell number. Endostatin protein's effect on cells early in the differentiation pathway results in a reduction of 70-80% of the final cell number. Endostatin protein also changes the morphology of these cells (data not shown).

### EXAMPLE 10

Co-localization of Endostatin Protein and Tropomyosin Protein

Incubation of HUVECs with Alexa Fluor™ 488 labeled recombinant human Endostatin protein (rhEndostatin) showed that once internalized the Endostatin protein displays a pattern of binding remarkably similar to that observed when the cells are immunostained for tropomyosin expression (FIG. 15A). The fluorescently labeled rhEndostatin protein localized to the microfilaments of the cell cytoskeleton in a pattern that overlapped with tropomyosin immunoreactivity. Double staining of HUVECs for both rhEndostatin-binding and tropomyosin expression demonstrated that tropomyosin immunoreactivity and Alexa Fluor™ 488 labeled rhEndostatin-binding co-localized to the microfilaments of the actin cytoskeleton (FIGS. 15D-E). Although not wanting to be bound by this theory, it is believed that rhEndostatin binding to tropomyosin disrupts actin microfilament function to activate endothelial cell apoptosis and thereby inhibit angiogenesis.

To determine whether live endothelial cells actively internalize Endostatin protein, viable HUVECs were incubated with Alexa Fluor™ 488 labeled rhEndostatin protein. The Alexa labeled rhEndostatin protein was rapidly internalized and translocated predominantly to the nucleus with weak cytoplasmic staining evident. Internalized rhEndostatin was rapidly processed such that by 60 minutes, little if any intercellular fluorescence was detected (data not shown). The rapid but transient transport of rhEndostatin to the nucleus suggests that the rhEndostatin protein is being processed in the nucleus.

Recombinant human Endostatin protein and hTM3 were also shown to interact in a capture ELISA (FIG. 8). The TM311 anti-tropomyosin monoclonal detected higher immunoreactivity in rhEndostatin coated wells than in wells coated with BSA to which the hTM3 expressing bacteria (λ21-1) lysate was added or in wells coated with rhEndostatin to which bacterial lysate containing no tropomyosin (control lysate) was incubated. This result confirms the binding of rhEndostatin to hTM3, an interaction that presumably occurs via the E37 mimotope of hTM3. Interestingly human cardiac tropomyosin, which lacks the E37 mimotope, failed to bind rhEndostatin protein in this assay (data not shown) supporting the assumption that the E37 mimotope of tropomyosin is required for rhEndostatin binding and that rhEndostatin does not interact with all forms of tropomyosin.

### EXAMPLE 11

Interaction of Laminin Protein and Angiostatin Protein

The following experiment was conducted in order to identify a potential receptor, or molecule, comprising a receptor or binding partner for Angiostatin protein. Peptides obtained by biopanning against Angiostatin protein were edited to remove multiple copies of sequences. This left the following sequences, arranged into specific classes of sequences. It is noted that the large number of lysines in the Angiostatin protein selected peptides is consistent with the known lysine-binding properties of plasminogen and plasminogen fragments containing intact kringle domains (lysine affinity is k4>k1>k2>k3).

TABLE 11
Linear Angiostatin Protein Binding Peptides

| Peptide | Amino Acid Sequence | SEQ ID NO: |
|---|---|---|
| PAL-49-M | KCCYYAK | SEQ ID NO:82 |
| PAL-50-M | KCCYSVG | SEQ ID NO:33 |
| PAL-51-M | KCCYPSA | SEQ ID NO:84 |
| PAL-56-M | HKYISAT | SEQ ID NO:85 |
| PAL-53-M | RQPPHLH | SEQ ID NO:86 |
| PAL-66-M | GTLQVLS | SEQ ID NO:87 |
| PAL-70-M | MSYQWSH | SEQ ID NO:38 |
| A1-M | ERVNDDT | SEQ ID NO:89 |
| A3-M | DRSGAIK | SEQ ID NO:90 |
| A7-M | LDRANVF | SEQ ID NO:91 |
| A9-M | SPLGGSE | SEQ ID NO:92 |
| A10-M | HAIYPRH | SEQ ID NO:93 |

TABLE 12
Cyclized Angiostatin Protein Binding Peptides

| Peptide | Amino Acid Sequence | SEQ ID NO: |
|---|---|---|
| PAC-77-M | C-WSLEFISK-C | SEQ ID NO:94 |
| A30-M | WTTJESTK | SEQ ID NO:95 |
| PAC-82-M | YTLPP-KL | SEQ ID NO:96 |
| PAC-88-M | WSYEWSK | SEQ ID NO:97 |
| PAC-91-M | WSLEWQK | SEQ ID NO:98 |
| A31-M | WSLEQSK | SEQ ID NO:99 |
| A28-M | WSLETTK | SEQ ID NO:100 |
| A33-M | WSLEHQK | SEQ ID NO:101 |
| PAC-78-M | VHSIERE | SEQ ID NO:102 |
| A32-M | GDMSDRP | SEQ ID NO:103 |

Based upon the sequences identified in Tables 11 and 12, searches were made for proteins containing the following sequences:

| | |
|---|---|
| WSLE | SEQ ID NO:104 |
| WTLE | SEQ ID NO:105 |
| WSYE | SEQ ID NO:106 |
| KCCY | SEQ ID NO:107 |
| TLQVL | SEQ ID NO:108 |

None of these motifs were present in fibrinogen, the natural substrate for plasminogen. However, there are a number of sequences in fibrinogen with significant homology to the selected peptides. These sequences are potentially involved in plasminogen binding and include:

| Fibrinogen alpha 91 | |
|---|---|
| SLFEYGK | SEQ ID NO:110 |
| WSLEWQK | SEQ ID NO:98 |
| Fibrinogen alpha 433 | |
| LVTSK | SEQ ID NO:111 |
| WSLETTK | SEQ ID NO:100 |
| WSYEWSK | SEQ ID NO:97 |
| WSLEQSK | SEQ ID NO:99 |
| WTLESTK | SEQ ID NO:95 |
| Fibrinogen alpha 477 | |
| EVTK | SEQ ID NO:112 |
| WSLETTK | SEQ ID NO:100 |
| WSYEWSK | SEQ ID NO:97 |
| WSLEQSK | SEQ ID NO:99 |
| WTLESTK | SEQ ID NO:95 |

Because of the presence of fibrinogen sequences with similarities to the selected peptides, additional data base searches were made and one, ERVN (SEQ ID NO:109) identified a tetrapeptide present in the laminin beta-1 chain. The laminin beta-1 chain is 1786 amino acids long and is one part of the laminin protein. The laminin protein is a heterotrimeric molecule with one each of the alpha, beta and gamma chains making up the molecule. The three chains form triple-helical coiled-coils at several places within the molecule. The amino acid sequence of the human laminin beta-1 protein is set forth in FIG. 6A (SEQ ID NO:113) and the nucleotide sequence is set forth in FIG. 6B (SEQ ID NO:114). The laminin beta-1 protein is a complex, multi-domain component of the basement membrane organized as follows:

| Amino Acid Range | Characterization |
|---|---|
| 1-21 | signal sequence |
| 22-270 | N-terminal domain |
| 271-540 | 4 = 1/2 EGF-like domains (4 complete, 1 partial) |
| 541-771 | laminin domain IV |
| 772-1178 | 8 EGF like domains |
| 1179-1397 | domain II |
| | 1216-1315 coiled-coil (approx 150 Angstroms long) |
| | 1353-1388 coiled-coil (approx 54 Angstroms long) |
| 1398-1430 | domain alpha |
| 1431-1786 | domain I |
| | 1442-1481 coiled-coil (approx 510 Angstroms long) |

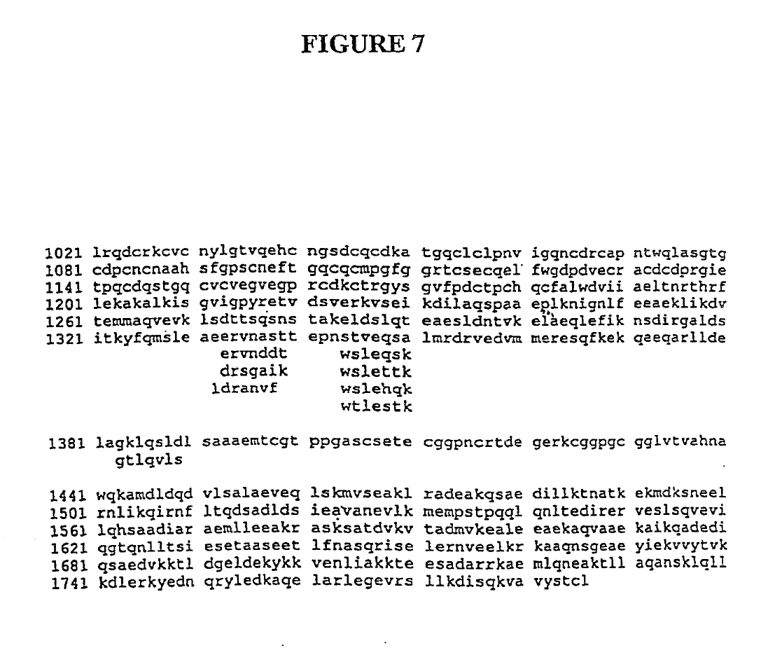

There are three motifs corresponding to Angiostatin protein binding peptides in a single 60 amino acid long region of laminin beta-1 chain designated as domain II. (See FIG. 7) The first two sites are in the part of domain II between the two coiled coils (the second binding site is just prior to the start of the second coiled coil). The third site is at the very end, but within, the second coiled coil region. Consequently, sites 2 and 3 are as much as 55 Angstroms apart, separated by most of the coiled coil. Therefore, the putative Angiostatin binding domains of the laminin beta-1 chain are found within the two coiled-coil regions of domain II. Additionally, since only the first amino acid of the first putative binding site is different in mouse laminin beta-1 chain, Angiostatin protein will most likely also bind to mouse laminin.

Using an antibody that recognizes laminin protein, the interaction of laminin protein with Angiostatin protein may be analyzed. Also, FACS analysis is conducted to demonstrate the location of laminin protein and to show its location on the surface of endothelial cells. Additional experiments blocking the binding of Angiostatin protein to the surface of HUVEC cells are performed. Further, immunoprecipitation of Angiostatin protein and a laminin beta-1 chain will confirm an association or binding between these proteins.

### EXAMPLE 12

### An Endostatin-binding Peptide shares an Epitope with hTM3

As detailed above, a cyclic (disulfide-bond constrained) random 7-mer peptide phage-display library was screened to identify peptides that interacted with rhEndostatin (Sim et al.). The cyclic, disulfide-bonded peptide, E37 (CTHWWHKRCGGGS) was found to bind rhEndostatin. A BLAST search of the National Center for Biotechnology Information database failed to identify proteins with any significant homology to the primary sequence of the E37 peptide. To identify proteins that share common epitope(s) with the E37 peptide, rabbit anti-sera was raised against KLH-coupled E37 peptide and used to probe an FGF-2 stimulated human umbilical vein endothelial cell (HUVEC) cDNA expression library. DNA sequencing of 9 clones recognized by E37 anti-sera identified 7 as being unique cDNAs of different lengths encoding non-muscle human tropomyosin isoform 3 (hTM3), suggesting that E37 and hTM3 share a common epitope.

A comparison of the primary sequence of hTM3 with that of the E37 peptide failed to reveal significant homology, suggesting that the E37 peptide sequence represents a conformational mimotope of the Endostatin-binding domain of tropomyosin. To confirm that the E37 peptide and human tropomyosin share an epitope, immunoblots of lysate from rhTM3 expressing E. coli (λ21-1) were probed with either TM311 anti-tropomyosin monoclonal antibody (Sigma, St Louis, Mo.) or anti-E37 polyclonal sera (FIG. 12). The anti-E37 sera and TM311 anti-tropomyosin antibody specifically recognized common bands of approximately 49 and 43 kDa in the rhTM3 expressing λ21-1 lysate whereas neither band was detected in immunoblots of control lysate (FIG. 12). Furthermore, when a lysate of HUVECs was probed with anti-E37 sera or TM311 anti-tropomyosin antibody, a common band of approximately 38 kDa was identified that co-migrated with E. coli expressed rhTM3 (data not shown). These findings demonstrate that the E37 peptide and hTM3 share a common epitope. The anti-E37 sera however failed to recognize tropomyosins purified from human cardiac tissue, rabbit muscle or chicken muscle (FIG. 12B). The amino acid sequences of rabbit muscle tropomyosin and human cardiac tropomyosin each share 86% identity with hTM3, diverging in two differentially spliced exons, exon 6 (amino acids 189 to 212) and exon 9 (amino acids 258 to 284). The inability of the anti-E37 sera to recognize either rabbit muscle or human cardiac tropomyosins suggests that the putative Endostatin-binding domain of hTM3 resides within either exon 6 or 9 of hTM3. Since the E37 peptide was identified based on its ability to bind rhEndostatin, the presence of the ‘E37 mimotope’ may define the specific isoforms of tropomyosin that bind Endostatin.

### EXAMPLE 13

### rhEndostatin and rhTM3 Interact in vitro

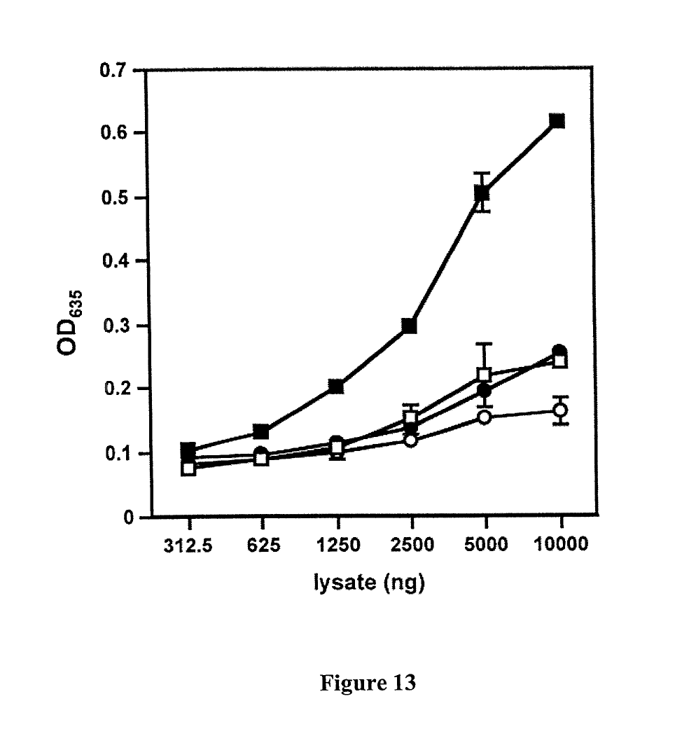

We characterized the interaction between rhEndostatin and tropomyosin by enzyme-linked immunosorbent assay (ELISA) and showed that rhEndostatin binds rhTM3-expressing bacterial lysate (λ21-1) (FIG. 13). This result provides evidence of a binding interaction between rhEndostatin and rhTM3.

Surface plasmon resonance performed using the BIAcore 3000 (BiaCore, Piscataway, N.J.) was used to assess the kinetics of rhEndostatin/rhTM3 binding. 3200 resonance units (RU) of purified rhTM3 were immobilized on the surface of a sensor chip via amine coupling and rhEndostatin injected over the chip as analyte. The rhEndostatin showed a dose dependent increase in RU, demonstrating the real-time binding of rhEndostatin to immobilized rhTM3 (FIG. 14A). The dissociation constant (K_{D}) for the rhEndostatin/rhTM3 interaction was calculated to be ˜100 μM using a steady state model. When, as a negative control, similar concentrations of rhAngiostatin K1-3 were passed over the rhTM3 chip, no evidence of specific binding to rhTM3 was observed (FIG. 14B), demonstrating the specificity of the rhEndostatin/rhTM3 interaction. To further evaluate the specificity of rhEndostatin/rhTM3 binding, competition experiments using soluble rhTM3 were performed. Soluble rhTM3 competed for the binding of rhEndostatin to a flow cell containing 2000 RU of immobilized rhTM3 in a dose-dependent manner (FIG. 14C), further demonstrating the specificity of the rhEndostatin/rhTM3 interaction.

### EXAMPLE 14

### Endostatin-binding and Tropomyosin Co-localize to Actin Microfilaments

Incubation of formalin fixed HUVECs, human microvascular endothelial cells (HMVECs) and human aortic endothelial cells (HAECs) with biologically active Alexa Fluor™ 488 labeled rhEndostatin showed that, given access to the cytosolic compartment, rhEndostatin displays a pattern of binding identical to that observed in cells immunostained for tropomyosin or filamentous actin (FIGS. 15A-C). Fluorescently labeled rhEndostatin and anti-tropomyosin antibodies co-localized to HUVEC microfilaments (FIGS. 15D-E). Similar results were obtained using anti-actin antibodies (data not shown). Thus, in fixed cells, Endostatin-binding, tropomyosin and actin co-localize to microfilament bundles of the cell cytoskeleton. We postulate that Endostatin, which is specifically internalized by endothelial cells Dixelius et al., binds tropomyosin to inhibit microfilament function, and as a consequence, tumor angiogenesis.

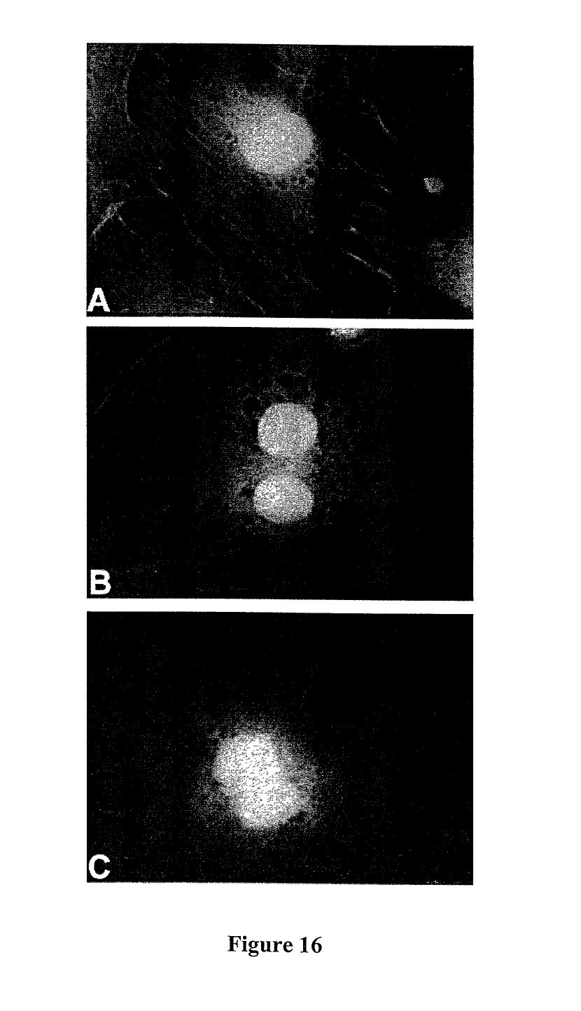

To determine if the binding of Alexa-labeled rhEndostatin to the HUVEC cytoskeleton was the result of an interaction with tropomyosin competition experiments were performed. A 50-fold molar excess of the E37 peptide, but not the control peptide inhibited the binding of rhEndostatin to HUVEC microfilaments (FIGS. 16A-C), thus demonstrating the role tropomyosin plays in Endostatin's binding the HUVEC cytoskeleton. This observation further confirms the role of the E37 mimotope plays in the interaction between Endostatin and hTM3.

### EXAMPLE 15

### E37 Peptide Blocks the Anti-metastatic Activity of rhEndostatin in vivo

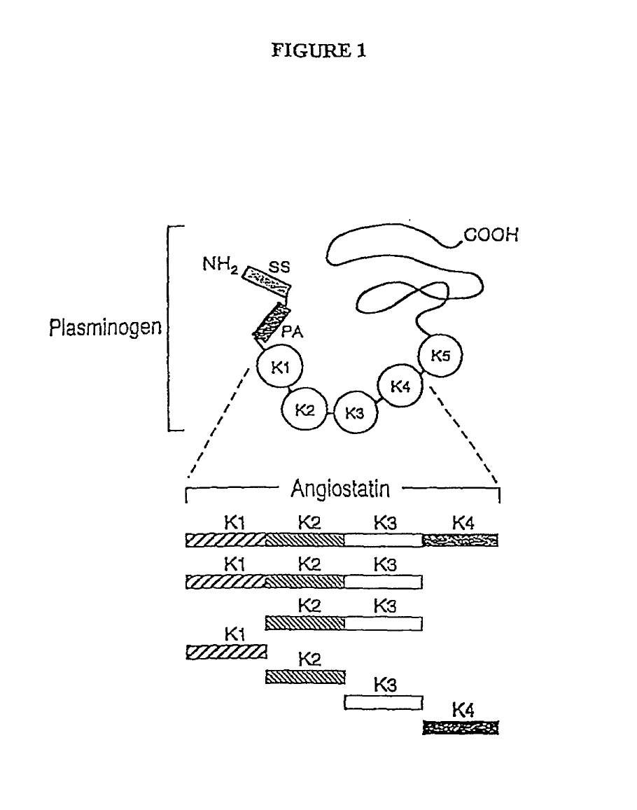

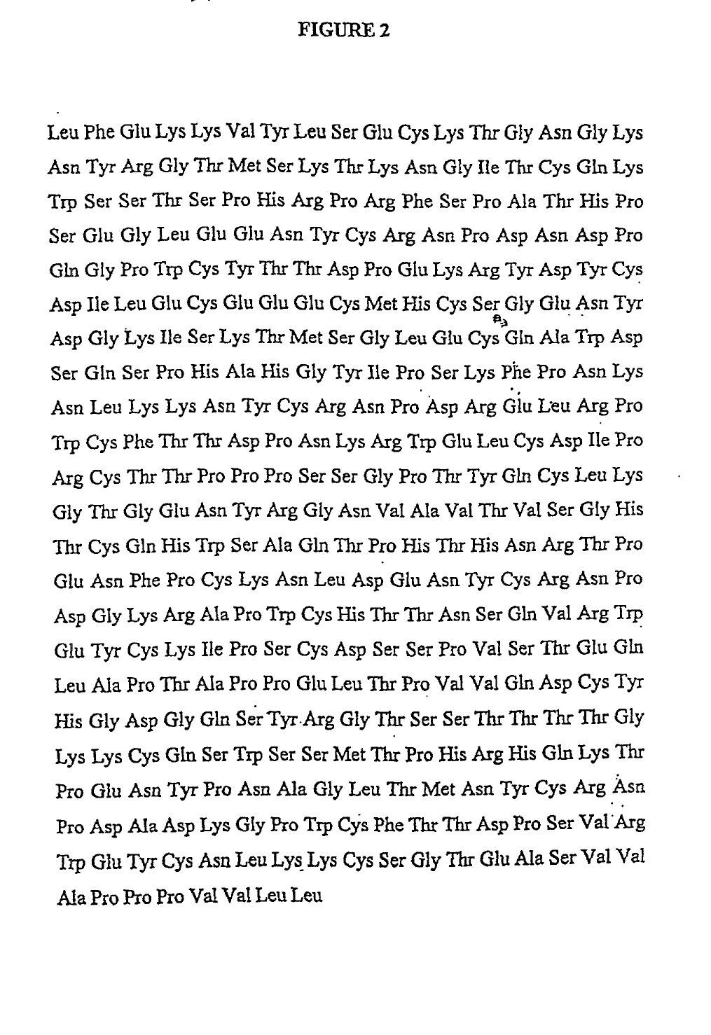

To further evaluate the biological significance of E37's interaction with Endostatin, we assessed the effect of the E37 peptide had on the anti-tumor activity of rhEndostatin. rhEndostatin has been shown previously to inhibit the growth of pulmonary metastases in the murine B16-BL6 experimental metastasis model in a dose-dependent manner (Sim et al.). Daily administration of 1.5 nmol of rhEndostatin (1.5 mg/kg/day) inhibited the growth of experimental B16-BL6 lung metastases by greater than 70% as compared to buffer-treated control mice (FIG. 1). The number of metastatic lesions on the lungs of the mice treated with up to 375 nmol/day of E37 peptide in the absence of rhEndostatin was not statistically different from that of the control group, demonstrating that the E37 peptide alone has neither tumor promoting or inhibitory activity in this assay. Co-administration of rhEndostatin with E37 peptide dramatically blocked the tumor growth inhibitory activity of the rhEndostatin (FIG. 17). Co-injection of 1.5 nmol of rhEndostatin with 15, 75 and 375 nmol of the E37 peptide resulted in a dose dependent decrease in the anti-tumor activity of the rhEndostatin of 11, 30.5 and 84.5 percent respectively. The number of pulmonary metastases in mice co-treated with rhEndostatin and either 75 or 375 nmol of E37 peptide (50 and 250-fold molar excess respectively) were significantly higher than those on the lungs of mice treated with either rhEndostatin alone or in combination with 75 or 375 nmol of control peptide respectively (P<0.02). The incidence of metastatic lesions on the lungs of mice co-injected with 1.5 nmol rhEndostatin and up to 375 nmol of a cyclic disulfide bonded control peptide, or in mice treated with 1.5 nmol of rhEndostatin alone, was statically indistinguishable (P>0.11). Thus, in the experimental B16-BL6 lung metastases model the anti-tumor activity of rhEndostatin is blocked in a dose-dependent manner by co-injection of E37 peptide. We conclude that because the E37 peptide mimics the binding pocket of TM3, E37 peptide competes with tropomyosin for rhEndostatin binding, resulting in disruption of the tropomyosin/rhEndostatin interaction and the rescue of angiogenesis and tumor growth.

An alternative explanation for E37 peptide's inhibitory activity is that co-administration of peptide with rhEndostatin results in perhaps the formation of an insoluble precipitate at the site of injection, leading to reduced bioavailability of rhEndostatin. To test this hypothesis, we measured sera concentrations of rhendostatin over time following subcutaneous (s.c.) administration of 1.5 nmol rhEndostatin alone or in combination with either 375 nmol (250-fold molar excess) of the E37 or control peptide. rhEndostatin co-administered with a 250-fold molar excess of E37 peptide had an area under the curveₗₐₛₜ (AUCₗₐₛₜ) equal to 85% of that observed when rhEndostatin was administered alone, or when rhEndostatin was dosed with 375 nmol of control peptide (Table 13). Assuming 100% bioavailability of rhEndostatin following its administration alone or with control peptide, then the bioavailable dose of rhEndostatin resulting from the co-administration of rhEndostatin and E37 peptide would correspond to 1.3 nmol (1.25 mg/kg). Inference from the B16-BL6 experimental metastasis model where a dose titration of rhEndostatin was used to inhibit metastatic growth (—Sim et al) suggested that a dose of 1.3 nmol would inhibit lung metastases by 77%. In this study, 1.5 nmol of rhEndostatin co-administered with a 250-fold molar excess of the E37 peptide resulted in an 11% inhibition of lung metastasis (FIG. 17). Thus, despite having a lower AUCₗₐₛₜ, reduction of rhEndostatin's activity in the B16-BL6 metastasis model by the E37 peptide cannot be attributed to decreased levels of circulating rhEndostatin. We thus conclude that the loss of tumor growth inhibition was caused by inactivation of rhEndostatin that results from the specific interaction of E37 with rhEndostatin.

### Table 13

### Bioavailability of rhEndostatin Administered in the Presence of E37 Peptide in Mice

Groups of mice (3 mice/time point) were injected s.c. with 1.5 nmol of rhEndostatin alone or in combination with a 250-fold molar excess of either E37 or control peptide. Sera were collected at 0.17, 0.5, 1 and 2 hours following s.c. administration, and assessed for serum rhEndostatin levels as previously described (Sim et al.)

AUCₗₐₛₜ (ng·min/ml)

| RhEndostatin | rhEndostatin + | rhEndostatin + |
|---|---|---|
| alone | 250x control peptide | 250x E37 peptide |
| 5370 | 5552 | 4544 |

It should be understood that the foregoing relates only to preferred embodiments of the present invention, and that numerous modifications or alterations may be made therein without departing from the spirit and the scope of the invention as set forth in the appended claims. The references cited throughout are hereby incorporated by reference in their entireties.

We claim:

1. A method of regulating angiogenesis in an individual comprising, administering to the individual an angiogenesis regulating amount of a pharmaceutical composition comprising a tropomyosin binding compound.

2. The method of claim 1, wherein the tropomyosin binding compound is selected from the group consisting of proteins or chemical compounds.

3. The method of claim 2, wherein the tropomyosin binding compound comprises tropomyosin isoforms.

4. The method of claim 3, wherein the tropomyosin isoform comprises an isoform selected from the group consisting of hTM1, hTM2, hTM3, hTM4, hTM5, hTM5a, hTM5b or hTMsma and active fragments thereof.

5. The method of claim 4, wherein the tropomyosin isoform comprises hTM3 and active fragments thereof.

6. The method of claim 1, wherein the tropomyosin binding compound binds to a non-muscle cell tropomyosin protein.

7. The method of claim 1, wherein the tropomyosin binding compound binds to a tropomyosin protein comprising, a span of amino acids substantially homologous to amino acids 189-212 of hTM3 (SEQ ID NO: 116), amino acids 258-284 (SEQ ID NO:117) of hTM3, or a combination thereof.

8. The method of claim 1, wherein the tropomyosin binding compound binds to a tropomyosin protein comprising, amino acids 189-212 of hTM3 (SEQ ID NO: 116), amino acids 258-284 (SEQ ID NO:117) of hTM3, or a combination thereof.

9. A method of inhibiting angiogenesis in an individual comprising, administering to the individual an angiogenesis inhibiting amount of a pharmaceutical composition comprising an actin disrupting compound.

10. The method of claim 9, wherein the actin disrupting compound is selected from the group consisting of proteins or chemical compounds

11. The method of claim 9, wherein the actin disrupting compound is a tropomyosin binding compound.

12. The method of claim 11, wherein the tropomyosin binding compound binds to a non-muscle cell tropomyosin protein.

13. The method of claim 11, wherein the tropomyosin binding compound comprises troposmyosin isoforms.

14. The method of claim 13, wherein the tropomyosin isoform comprises an isoform selected from the group consisting of hTM1, hTM2, hTM3, hTM4, hTM5, hTM5a, hTM5b or hTMsma and active fragments thereof.

15. The method of claim 14, wherein the tropomyosin isoform comprises hTM3 and active fragments thereof.

16. The method of claim 15, wherein the tropomyosin binding compound binds to a tropomyosin protein comprising, a span of amino acids substantially homologous to amino acids 189-212 of hTM3—(SEQ ID NO: 116), amino acids 258-284 (SEQ ID NO:117) of hTM3, or a combination thereof.

17. The method of claim 16, wherein the actin disrupting compound comprises amino acids 189-212 of hTM3 (SEQ ID NO: 116), amino acids 258-284 (SEQ ID NO:117) of hTM3, or a combination thereof.

18. The method of claim 16, wherein the tropomyosin binding compound binds to a tropomyosin protein comprising, amino acids 189-212 of hTM3, amino acids 258-284 of hTM3, or a combination thereof.
